SEQUENCE LISTING (9 sequences)

SEQ ID NO: 1 - DNA, 21 bp, Artificial sequence
  Synthetic
  misc_feature (1)..(21): DNA/RNA Hybrid
acagcaaauuccaucgugutt

SEQ ID NO: 2 - DNA, 21 bp, Artificial sequence
  Synthetic
  misc_feature (1)..(21): DNA/RNA Hybrid
acacgauggaauuugcugutt

SEQ ID NO: 3 - DNA, 21 bp, Artificial sequence
  Synthetic
  misc_feature (1)..(21): DNA/RNA Hybrid
acagcaaauuccaucgugutt

SEQ ID NO: 4 - DNA, 21 bp, Artificial sequence
  Synthetic
  misc_feature (1)..(21): DNA/RNA Hybrid
gcgcgcuuuguaggauucgtt

SEQ ID NO: 5 - DNA, 21 bp, Artificial sequence
  Synthetic
  misc_feature (1)..(21): DNA/RNA Hybrid
cgaauccuacaaagcgcgctt

SEQ ID NO: 6 - DNA, 20 bp, Artificial sequence
  Synthetic
gatggcacaggaggaaagag

SEQ ID NO: 7 - DNA, 20 bp, Artificial sequence
  Synthetic
agccaggctgtcttgactgt

SEQ ID NO: 8 - DNA, 20 bp, Artificial sequence
  Synthetic
gtctgtgatgcccttagatg

SEQ ID NO: 9 - DNA, 20 bp, Artificial sequence
  Synthetic
agcttatgacccgcacttac

(19) United States
(12) Patent Application Publication
(10) Pub. No.: US 2022/0107246 A1
(43) Pub. Date: Apr. 7, 2022

(54) NONCOVALENT TAGS AND RELATED METHODS FOR CYTOSOLIC DELIVERY OF MACROBIOMOLECULES

(71) Applicant: University of Washington, Seattle, WA (US)

(72) Inventors: Xiaohu Gao, Seattle, WA (US); Wanyi Tai, Seattle, WA (US)

(73) Assignee: University of Washington, Seattle, WA (US)

(21) Appl. No.: 17/426,448

(22) PCT Filed: Jan. 31, 2020
(86) PCT No.: PCT/US2020/016256

§ 371 Date: Jul. 28, 2021

Related U.S. Application Data
(60) Provisional application No. 62/800,093, filed on Feb. 1, 2019.

(51) Int. Cl. G01N 1/30; C12N 15/113

(52) U.S. Cl. CPC G01N 1/30; C12N 15/113; G01N 2001/302; C12N 2310/14; C12N 2310/53; C12N 2310/111

(57) ABSTRACT

The disclosure provides bifunctional tag constructs and related methods useful for transporting macrobiomolecules, such as nucleic acids and proteins, across plasma membranes. The bifunctional tags comprise an affinity domain conjugated to hydrophobic domain. The affinity domain is configured to noncovalently bind to the macrobiomolecule, whereas the hydrophobic domain is configured to interact with the plasma membrane. In certain embodiments, the plurality of bifunctional tags can noncovalently associate along the length of a macrobiomolecule, thus increasing the interaction with and penetration through the plasma membrane.

### CROSS-REFERENCE TO RELATED APPLICATION

This application claims the benefit of U.S. Provisional Patent Application No. 62/800,093, filed Feb. 1, 2019, which is incorporated herein by reference in its entirety.

### STATEMENT OF GOVERNMENT LICENSE RIGHTS

This invention was made with government support under Grant No. R01 CA140295, awarded by the National Institutes of Health. The government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 70830_Seq_final_2020-01-22.txt. The text file is 3 KB; was created on Jan. 22, 2020; and is being submitted via EFS-Web with the filing of the specification.

### BACKGROUND

Living cells are the basic building blocks of the body, are spatially and temporally complex within organisms, and perform a diverse spectrum of functions including growth, mass transport, energy production, metabolism, and reproduction. These functions are generally encoded by the DNAs and RNAs and realized by proteins. Modulating intra-cellular activities provides a direct means to precisely control many biological processes and the associated human diseases. In principle, these ambitions in biology and medicine can be readily achieved with nucleic acid and peptide/protein-based reagents and therapeutics. For example, immunological agents such as antibodies and antibody fragments have been widely explored, because hundreds of their broad availability and accessible modes of development. In addition, immunological agents can facilitate dynamic monitoring of the protein activities with a cell and allow an in-depth understanding of the nature of cell physiology and pathology.

For example, in fluorescence microscopy, tagging biomarkers of interest with fluorescently labeled antibodies allows imaging biomarker localization, co-localization, translocation, and expression levels with high sensitivity, high spatial and temporal resolution. Similarly, in drug development, new therapeutics based on immunological agents are becoming increasingly attractive. With a higher degree of complexity, protein therapeutics offer tunable binding affinity, improved binding specificity, and lower side effects compared to many small-molecule drugs, promising a paradigm shift in both drug discovery and disease treatment. It is worth mentioning that biological techniques for raising antibodies, nanobodies, or screening functional peptides (e.g., hybridoma and phage display) have matured over the years. Since the early 1980s, over 230 therapeutic proteins (including peptides) and their 380 drug variants have received regulatory approval for human uses.

However, despite this rapidly increasing approved immunological-based drugs, the field is still missing out on the majority of cell signaling nodes and drug targets inside the cells. The fundamental limitation of the current immunological diagnosis and interventions is their incapability of interrogating or modulating intracellular targets in live cells. Considering the three dimensional structure of cells, there are far more intracellular targets with biological significance than their cell membrane counterparts. For immunofluorescence microscopy, cells have to be fixed first, thus only providing a snapshot of the dynamic and evolving cell signaling process. For therapeutics, hydrophilic macromolecular protein drugs (e.g., antibodies, Fc fusions, hormones, cytokines, and enzymes) that cannot cross the cell membrane spontaneously, only act on the limited number of extracellular targets.

The significance and urgent need of intracellular delivery of active biological agents (e.g., macrobiomolecules) are widely recognized in biology and medicine.

A variety of technologies have been developed in the past several decades that partially address this chronic problem. For example, microinjection can inject any biologics directly into the cytosol but is of very low throughput (one cell at a time). High-throughput approaches to punch transient pores in cell membrane have also been developed, based on electroporation, toxin, hypertonic solution, and mechanical forces. Compromising the cell membrane integrity allows material exchange between the cytoplasm and the outside media, but it is also toxic to cells and affects cell normal physiology. Another popular category of technologies for intracellular delivery of biologics uses chemical agents such as liposomes, polymers, and cell-penetrating peptides (CPPs). These compounds do not punch holes in the cell membrane, but a common issue shared by these technologies is that cargo (e.g., protein molecules) are predominantly delivered inside cells via endocytosis, a remarkably effective cell defense mechanism to prevent intact foreign biomolecules to enter the cytoplasm. As a result, the vast majority of ingested macrobiomolecules are degraded inside endosomes and lysosomes, limiting the bioavailability of the imaging or therapeutic biological agent.

Many of the challenges faced in intracellular delivery of proteinaceous macrobiomolecules also apply to nucleic acid-based macrobiomolecules. For example, short interference RNAs (siRNAs) are small duplexed RNA molecules capable of controlling post-transcriptional gene expression. They have the potential to create a new paradigm in disease intervention and management because they i) can control gene expression products that are considered undruggable, ii) can be designed following the simple base-paring rule for individual patients and swiftly tweaked according to patients' disease developments, and iii) can be delivered by the same carrier independent of the sequence (unlike conventional drugs with different pharmacokinetic and pharmacodynamic profiles). Despite these unique advantages, siRNA is far from reaching its full therapeutic potential. Significant research efforts in the past two decades have produced a number of siRNA formulations for clinical trials, yet the delivery efficiencies are still very limited. A major difficulty is how to overcome the cellular barrier, efficiently carrying siRNA into cytosol where RNAi takes place.

Unlike small (<1 kDa), charge-neutral, and hydrophobic molecules that can passively diffuse across the cellular membrane, siRNAs are highly anionic macrobiomolecules (˜14 kDa) and thus cannot enter cells by themselves. To address this issue, a number of carriers have been developed, in particular, cationic lipids and polymers that are not only able to condense DNA and RNA into nanoparticles, but also aid in endosome escape when they are taken up by cells. Despite decades of research and development, these conventional cationic materials are losing traction towards clinical uses due their poor penetration into solid tissues, colloidal instability in circulation, and toxicity. Instead, siRNA delivery has shifted towards precisely defined molecular conjugates, such as by chemically modifying siRNA molecules with hydrophobic moieties. One of the most noticeable achievements in this arena is siRNA terminal modification with a cholesterol molecule (either at the 5′ or 3′ end of the sense strand). When incubated with cells, the amphiphilic cholesterol-siRNA conjugate (chol-siRNA) fuses into the cell plasma membrane and enter cells via bulk-phase pinocytosis. Similarly, siRNA prodrugs (phosphates converted to charge-neutral phosphotriesters) have been developed, which can be hydrolyzed back to native siRNA after entering cells. Towards clinical translation, a pipeline of siRNA conjugates such as siRNA-N-Acetylgalactosamine (GalNAc) is under late-stage clinical trials. However, despite these exciting advances, the vast majority of the internalized siRNA are still trapped in lysosomes and other oligonucleotide sinks. The exceedingly low percentage of siRNAs escaping from endosomes (often <1%), significantly limits the potential of RNA-based therapeutics (similar for other biologics).

Accordingly, despite the advances in the fields of macrobiologics and cytosolic delivery of such macrobiomolecule agents, there remains a need for facile and efficient intracellular, e.g., cytosolic, delivery of the macrobiomolecule agents that can be applied broadly across the spectrum of potentially beneficial therapeutics and reagents. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a bifunctional tag. The bifunctional tag comprises an affinity domain conjugated to a hydrophobic domain. The affinity domain is configured to non-covalently bind a macrobiomolecule and the hydrophobic domain is configured to interact with a plasma membrane.

In some embodiments, the macrobiomolecule is or comprises a nucleic acid. In some embodiments, the nucleic acid is double stranded. In some embodiments, the double-stranded nucleic acid is DNA. In some embodiments, the double-stranded nucleic acid is double-stranded RNA. In some embodiments, the double-stranded RNA is a small interfering RNA (siRNA) or a microRNA (miRNA). In some embodiments, the affinity domain is or comprises a nucleic acid binding agent. In some embodiments, the affinity domain is or comprises an intercalating agent. In some embodiments, the affinity domain is or comprises ethidium bromide, acridine, bleomycin, doxorubicin, carbocyanine, DAPI, hoechst, distamycin, metropsin, cisplatin, and the like.

In some embodiments, the macrobiomolecule is or comprises a peptide, polypeptide, or protein. In some embodiments, the polypeptide or protein is an antibody, antibody derivative, an enzyme, a cytokine, or hormone. In some embodiments, the antibody derivative comprises an antibody fragment. In some embodiments, the antibody derivative is a single-chain Fv (scFv) or a single-domain antibody (sdAb or nanobody). In some embodiments, the antibody is a chimeric antibody, a humanized antibody, or a fragment thereof. In some embodiments, the affinity domain comprises a peptide, polypeptide, or protein binding agent. In some embodiments, the peptide, polypeptide, or protein staining agent is Coomassie blue, and the like.

In some embodiments, the hydrophobic domain is or comprises a linear or cyclic hydrocarbon structure. In some embodiments, the hydrocarbon has between 6 and about 50 carbons. In some embodiments, the hydrophobic domain is or comprises a steroid. In some embodiments, the steroid is cholesterol or a derivative thereof.

In some embodiments, the affinity domain and the hydrophobic domain are covalently conjugated by a linker domain. The linker domain can be linear and conjugate a single affinity domain to a single hydrophobic domain. Alternatively, the linker domain can be branched and conjugate a single affinity domain to a plurality of hydrophobic domains, conjugate a plurality of affinity domains to a single hydrophobic domain, or conjugate a plurality of affinity domains to a plurality of hydrophobic domains.

In some embodiments, the bifunctional tag is configured to disassociate from the macrobiomolecule upon passage of the macrobiomolecule through the plasma membrane.

In another aspect, the disclosure provides a composition comprising a plurality of bifunctional tags as described herein, a macrobiomolecule, and an acceptable carrier, wherein the plurality of bifunctional tags are non-covalently bound to the macrobiomolecule by their respective affinity domains. In some embodiments, the carrier is a pharmaceutically acceptable carrier.

In another aspect, the disclosure provides a method of delivering a macrobiomolecule to the cytosol of a cell, comprising contacting the cell with a macrobiomolecule noncovalently bound by at least one bifunctional tag as described herein. In some embodiments, the macrobiomolecule is bound by a plurality of bifunctional tags. In some embodiments, the macrobiomolecule and bifunctional tags are present at a molar ratio of at least about 1:3, at least about 1:4, at least about 1:5, at least about 1:6, at least about 1:7, at least about 1:8, at least about 1:9, at least about 1:10, at least about 1:15, at least about 1:20, at least about 1:25, at least about 1:30, or at least about 1:40. In some embodiments, the macrobiomolecule has a molecular weight of between about 0.5 kDa and about 250 kDa. In some embodiments, the biomedical molecule has a molecular weight of between about 1 kDa and about 100 kDa, between about 1 kDa and about 75 kDa, between about 1 kDa and about 60 kDa, between about 1 kDa and about 50 kDa, between 1 kDa and about 40 kDa, between about 1 kDa and about 30 kDa, or between about 1 kDa and about 25 kDa. In some embodiments, the macromolecule has a molecular weight of between about 1 kDa and about 50 kDa. In some embodiments (e.g., wherein the macrobiomolecule is less than 50 kDa, 40 kDa, etc.) the macromolecule is delivered to the cytosol by direct permeation through the plasma membrane. In some embodiments, the macromolecule is delivered to the cytosol by endocytosis. For example, in some embodiments where the macromolecule delivered by endocytosis, the macromolecule has a molecular weight of between about 40 kDa and about 250 kDa.

The cell can be in in vitro culture or alternatively in vivo in a subject. In some embodiments wherein the cell is in vivo in a subject, a therapeutically effective amount of the macrobiomolecule noncovalently bound by at least one bifunctional tag as described herein is administered to the subject in need thereof. The subject can be, e.g., a mammal, such as human, nonhuman primate, mouse, rat, guinea pig, rabbit, cat, dog, sheep, horse, cow, and the like.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A and 1B are schematic illustrations of siRNA modified with hydrophobic compounds and its cell entry. (FIG. 1A) Covalently linked hydrophobic ligand-siRNA enters cells via endocytosis and pinocytosis. (FIG. 1B) siRNA non-covalently tagged with multiple hydrophobic ligands directly slips into the cytosol.

FIGS. 2A-2H illustrate tag design and characterizations. (FIG. 2A) Intercalation of conventional DNA intercalators into siRNA monitored by fluorescence activation. Ethidium and acridinium show similar fluorescence activation in both DNA and RNA, unlike the others. (FIG. 2B) Gel electrophoresis of siRNA with acridinium and ethidium at low and high degree of intercalation. Acridinium unwinds siRNA duplex to form red-fluorescent complex at loading well at high degree of intercalation, while ethidium does not. (FIG. 2C) Screening hydrophobic compounds (after linking with ethidium) for mRNA (PPIB) knockdown in PC-3 cells. The numbers represent the compounds illustrated in FIG. 2D. Steroids such as cholesterol and stigmasterol offer the highest RNAi efficiency. (FIG. 2D) Molecular structures of the 14 exemplary chemical tags tested in the current study. The hydrophobic compounds are linked to ethidium via a C₆ spacer. (FIG. 2E) Dose response of gene knockdown using siRNA tagged with ethidium-cholesterol (left) in PC-3 cells. (FIG. 2F) RNAi efficiency of the tagged siRNA in the presence (w/) or absence (w/o) of serum. (FIG. 2G) Albumin binding assay for the tagged siRNA (incubation in 2.0 mg/mL BSA followed by agarose gel electrophoresis. (FIG. 2H) Fluorimetric titration curve for siRNA titrated with various amounts of tag 5. A IF: the fluorescent enhancement.

FIGS. 3A and 3B graphically illustrate confirmation of siRNA cytosolic delivery mechanism. (FIG. 3A) Quantitative flow cytometry studies of cellular uptake of chol-siRNA and the tagged siRNA on PC-3 cells after 2-hour incubation at 37° C. (FIG. 3B) Cell uptake of the tagged siRNA is temperature dependent, but not impaired by various endocytosis inhibitors. Baf. A, bafilomycin A; Cyt. B, cytochalasin B; and Dyn, dynasore.

FIG. 4 graphically illustrates an in vitro dose-dependent cytotoxicity study of the tagged siRNA (scrambled control siRNA) on PC-3 cells.

FIGS. 5A-5D illustrate in vivo delivery of the tagged siRNA to the liver. (FIG. 5A) In vivo biodistribution of free and tagged siRNA (Cy5.5 labeled) monitored by NIR imaging 1- and 8-h post injection. LU: lung, LI: liver, IT: intestine, UB: urine bladder. (FIG. 5B) Ex vivo comparison of liver siRNA accumulation of free siRNA and tagged siRNA 8-h post injection. (FIG. 5C) Fluorescence quantification of the images shown in panel b (normalized by liver mass), P<0.05. (FIG. 5D) Gene knockdown in the liver after a single intravenous injection of the free siRNA and tagged siRNA. P<0.05.

FIG. 6 is a chemical schematic illustrating synthesis of various illustrative small-molecule tags encompassed by the present disclosure.

FIG. 7 illustrates chemical structures of exemplary DNA intercalators tested in the current study disclosed herein.

FIG. 8 graphically illustrates intercalation of tag 5, 9, and 14 into siRNA monitored with fluorescence activation. Compared to tag 5, tags 9 and 14 only show minor fluorescence enhancement indicating weak binding with siRNA.

FIGS. 9A and 9B illustrate successful cytosolic delivery of protein by cholesterol tagging. (FIG. 9A) The protein delivery tag is designed by joining two copies of cholesterol with a Coomassie blue G250 molecule via an aminated crosslinker. (FIG. 9B) Upon mixing with proteins, the CB end anchors onto the protein surface, whereas the exposed cholesterol makes the cargo protein compatible with the cell membrane lipid bilayers. The protein-tag complex embedded between the bilayers eventually dissociates due to the non-covalent nature of the protein-tag binding, leaving a hydrophilic macromolecule in the hydrophobic bilayer. This incompatibility renders the protein molecule to slip out of the membrane, either into the extracellular space or directly into the cytosol avoiding endocytic sequestration.

FIGS. 10A and 10B schematically illustrate the effect of tag density on delivery and confirmation of delivery route. High tag/protein ratio produces sufficient cholesterol coverage of protein surface and favors direct membrane permeation (FIG. 10A), whereas insufficient cholesterol coverage leads to initial protein anchoring on cell surface followed by endocytosis (FIG. 10B).

FIG. 11 illustrates the delivery of Cyt c, a model intracellular protein drug, into living tumor cells using our tag causing apoptosis. Cyt c-triggered cell apoptosis. Dose-dependent cell apoptosis was observed only in tagged Cyt c, but not Cyt c alone (average/s.d. from 6 measurements).

FIGS. 12A and 12B schematically show receptor-mediated endocytosis (FIG. 12A) and the endocytosis of proteins under-tagged than the optimal tag/protein ratio (FIG. 12B).

FIG. 13 graphically illustrates confirmation of protein function after non-covalent cholesterol tagging The graph shows a comparison of the enzymatic activity of HRP and cholesterol tagged HRP demonstrating that virtually no change of fluorescence or enzymatic activity was observed.

FIG. 14 graphically illustrates the toxicity evaluation of CB and cholesterol tag on HeLa cells by AlamarBlue assay.

FIG. 15 schematically illustrates the synthetic route of the CB-cholesterol tag (5). Cholesteryl chloroformate (1) was reacted with excess amount (5 equivalents) of the linker (2) to obtain aminated cholesterol (3). Two copies of (3) were conjugated to Coomassie blue G250, and the tertiary amine of the linker was methylated to quaternary amine.

### DETAILED DESCRIPTION

Macrobiomolecules such as proteins and nucleic acids offer the potential to tag intracellular structures and modulate a wide variety of cellular processes in a targeted and specific manner. However, as described above, the current technologies are limited by the efficiency and delivery of larger molecules into the cytosol of living cells.

To illustrate, short interfering RNA (siRNA) has broad applications in biology and medicine, and holds tremendous potential to become a new class of therapeutics for many diseases. As a highly anionic macrobiomolecule, its cytosolic delivery, however, has been a major roadblock in translation. Furthermore, protein-based imaging agents and therapeutics are superior in structural and functional diversity compared to small molecules, and more importantly, much easier to design or screen. For example, an antibody or antibody fragment can be easily raised against virtually any target, promising great potential for cell signaling studies and drug discovery. Despite these fundamental advantages, the power and impact of protein-based agents are substantially undermined, only acting on a limited number of extracellular targets because hydrophilic macromolecules cannot spontaneously cross the cell membrane. Conventional protein delivery techniques fail to address this fundamental problem in that protein cargos are predominantly delivered inside cells via endocytosis, a remarkably effective cell defense mechanism naturally evolved to prevent intact biomolecules from entering the cytoplasm.

This disclosure describes the development of a bifunctional tag strategy to non-covalently tag macrobiomolecules to enhance their ability to interact with the plasma membrane and permeate therethrough, thereby facilitating efficient delivery to the cytosol of a cell. As described in more detail below, in one study small, bifunctional chemical tags capable of transporting nucleic acids directly into the cytosol were developed and characterized. Briefly, bifunctional tags consist of a nucleic acid-binding moiety that interacts with the nucleic acid (i.e., siRNA) non-covalently, and a steroid domain that readily fuses with the mammalian cell membrane. In contrast to the conventional covalently conjugated siRNA-steroid conjugate that enters cells largely via endocytosis which substantially limits siRNA bioavailability, the non-covalently tagged siRNA is cell membrane-permeant, avoiding the endocytic pathway. This new methodology enables, for example, effective RNA interference (RNAi) without the need of cationic transfection or endosomolytic agents, opening a new avenue for intracellular delivery of native biologics. In a second study, described in more detail below, the bifunctional tag design was modified and extended to successfully tag protein payloads for cytosolic delivery, thus enabling virtually any compact proteins to permeate through the cell plasma membrane, completely bypassing endocytosis. This simple plug-and-play platform greatly expands the biological target space and has the potential to transform basic biology studies and drug discovery.

In accordance with the foregoing, the present disclosure provides a bifunctional tag that comprises an affinity domain conjugated to a hydrophobic domain. The affinity domain is configured to non-covalently bind a macrobiomolecule and the hydrophobic domain is configured to interact with a plasma membrane.

As used herein, the term “macrobiomolecule” generally refers to large molecules obtained from or derived from biological sources, such as cells or tissues, and can perform biological processes. Exemplary macrobiomolecules encompassed by the present disclosure are nucleic acids and peptides/polypeptides/proteins, or combinations thereof. The bifunctional tags described herein can be applicable to any macrobiomolecule without limitation. In the context of the present disclosure, the macrobiomolecule can be considered a payload, and is periodically referred herein to as such. As described in more detail below the size of the payload can influence the manner in which macrobiomolecule payloads are actually delivered into the interior the cell e.g. direct permeation through the plasma membrane versus endocytosis. In some embodiments, the macrobiomolecule can be between about 0.5 kDa and about 250 kDa.

In one embodiment, the macrobiomolecule is a nucleic acid. As used herein, the term “nucleic acid” refers to a deoxyribonucleotide polymer (DNA) or ribonucleotide polymer (RNA) in either single- or double-stranded form. The structure of the canonical polymer subunits of DNA, for example, are commonly known and are referred to herein as adenine (A), guanine (G), cytosine (C), and thymine (T). As a group, these are generally referred to herein as nucleotides or nucleotide residues. For RNA, the four canonical polymer subunits are the same, except with uracil (U) instead of thymine (T). The nucleic acids encompassed by the present disclosure can include deoxyribonucleotide polymer (DNA), ribonucleotide polymer (RNA), cDNA, or a synthetic nucleic acid known in the art. In some embodiments, the nucleic acid is double stranded. The double-stranded nucleic acid can be DNA, or in other embodiments, the double-stranded nucleic acid is double-stranded RNA. Exemplary double-stranded RNA constructs can include, but are not limited to, small interfering RNA (siRNA) or a microRNA (miRNA) constructs. These constructs can facilitate RNAi and can readily be configured to target any target gene transcript of choice for degradation.

In these embodiments where the macrobiomolecule is a nucleic acid, the affinity domain is or comprises a molecular construct or moiety that can non-covalently bind to the selected nucleic acid macrobiomolecule. Such an affinity domain can be referred to as a “nucleic acid binding agent”. Typically, the affinity domain is not specific for noncovalently binding to any particular nucleic acid sequence, but rather can generally bind to nucleic acids, or at least general types of nucleic acids (e.g., double-stranded DNA, double-stranded RNA, and the like) regardless of specific sequence. A wide variety of nucleic acid binding agents are known in the art and are encompassed by the present disclosure. For example, some nucleic acid binding agents are nucleic acid stains that have heretofore been used to image or provide detectable signals associated with nucleic acids. In the context of the present disclosure, any detectable signal is ancillary to the primary function of being able to form noncovalent associations or bonds with nucleic acid-containing payloads. In some embodiments, the affinity domain is or comprises an intercalating agent. For example, illustrative nonlimiting intercalators useful for binding DNA include acridine, ethidium (e.g. ethidium bromide), doxorubicin, carbocyanine compound, and the like. Additional affinity domains that can noncovalently bind DNA include minor groove binders. Illustrative, non-limiting examples of DNA minor groove binders include compounds such as 4′,6-diamidino-2-phenylindole (DAPI), Hoechst dyes, distamycin, netropsin and the like. Additional affinity domains that can noncovalently bind DNA are cross-linker agents, such as cisplatin, and the like. As described in more detail below intercalating compounds can also noncovalently bind to RNA constructs, for example when the RNA is double-stranded. Exemplary RNA binding agents encompassed by the present application include ethidium (e.g., ethidium bromide) acridine, and bleomycin. However, it will be noted that the exemplary nucleic acid binding agents described above are not limiting. Persons of ordinary skill in the art readily incorporates other nucleic acid binding agents available in the art into the present disclosure.

In other embodiments, the macrobiomolecule is or comprises a peptide, polypeptide, or protein.

As used herein, the term “peptide” refers to a short chain of multiple amino acid subunits linked by amide bonds. As used herein, the term “polypeptide” or “protein” refers to a longer polymer in which amino acid residues that are joined together through amide bonds. Peptides typically refers to shorter length amino acid polymers, for example polymers of less than 50 amino acids in length. Polypeptide can include longer amino acid polymers. A protein addresses embodiments that can be one or multiple polypeptides arranged in a biologically functional way. When the amino acids are alpha-amino acids, either the L-optical isomer or the D-optical isomer can be used, the L-isomers being preferred. The terms “peptide”, “polypeptide”, or “protein” as used herein encompasses any amino acid sequence and includes modified sequences such as glycoproteins. The term polypeptide is specifically intended to cover naturally occurring proteins, as well as those that are recombinantly or synthetically produced. All peptide, polypeptide, or protein macrobiomolecules are encompassed herein as potential payloads without limitation. As indicated herein, the size of the peptide, polypeptide, protein payload is not limited. In some embodiments, the size of the peptide, polypeptide, protein payload is between about 0.5 kDa and about 250 kDa, as described in more detail herein. Additionally, the identity or type of peptide, polypeptide, or protein macrobiomolecule is not limited, but rather can be any peptide, polypeptide, or protein macrobiomolecule. Illustrative, nonlimiting examples of polypeptide or protein macrobiomolecules encompassed by the present disclosure include affinity reagents (e.g., antibodies and antibody derivatives), enzyme, cytokines, hormone, and the like.

As used herein, the term “antibody” encompasses immunoglobulin molecules produced by or derived from any antibody-producing mammal (e.g., mouse, rat, rabbit, and primate including human), and which specifically bind to an antigen of interest (e.g., an intra-cellular antigen). Exemplary antibodies include polyclonal, monoclonal and recombinant antibodies; multispecific antibodies (e.g., bispecific antibodies); humanized antibodies; murine antibodies; and chimeric antibodies, such as mouse-human, mouse-primate, primate-human monoclonal antibodies, and the like.

The term antibody derivative encompasses fragments, modifications, fusions, or other constructs that incorporate structure of at least part of an antibody molecule. An antibody fragment is a portion derived from or related to a full-length antibody, including the complementarity-determining regions (CDRs), antigen binding regions, or variable regions thereof. Illustrative examples of antibody fragments useful in the present disclosure include Fab, Fab′, F(ab)₂, F(ab′)₂ and Fv fragments, VHH fragment, VNAR fragment, scFv fragments, single-chain Fab fragment (scFab), diabodies, linear antibodies, single-chain antibody molecules, multi specific antibodies formed from antibody fragments, and the like. A “single-chain Fv” or “scFv” antibody fragment comprises the V_{H} and V_{L} domains of an antibody, wherein these domains are present in a single polypeptide chain. The Fv polypeptide can further comprise a polypeptide linker between the V_{H} and V_{L} domains, which enables the scFv to form the desired structure for antigen binding.

Antibodies can be further modified to suit various uses. For example, a “chimeric antibody” is a recombinant protein that contains domains from different sources. For example, the variable domains and complementarity-determining regions (CDRs) can be derived from a non-human species (e.g., rodent) antibody, while the remainder of the antibody molecule is derived from a human antibody. A “humanized antibody” is a chimeric antibody that comprises a minimal sequence that conforms to specific complementarity-determining regions derived from non-human immunoglobulin that is transplanted into a human antibody framework.

Antibody fragments and derivatives that recognize specific epitopes can be generated by any technique known to those of skill in the art. For example, Fab and F(ab′)₂ fragments of the invention can be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CHI domain of the heavy chain. Further, the antibodies of the present invention can also be generated using various phage display methods known in the art. Finally, the antibodies, or antibody fragments or derivatives can be produced recombinantly according to known techniques.

Production of antibodies or antibody derivatives can be accomplished using any technique commonly known in the art. Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual (Cold Spring Harbor Laboratory Press, 2nd ed. 1988); Hammerling et al., in: Monoclonal Antibodies and T-Cell Hybridomas 563-681 (Elsevier, N.Y., 1981), incorporated herein by reference in their entireties. The term “monoclonal antibody” refers to an antibody that is derived from a single clone, including any eukaryotic, prokaryotic, or phage clone, and not the method by which it is produced. Methods for producing and screening for specific antibodies using hybridoma technology are routine and well-known in the art. Once a monoclonal antibody is identified, the encoding gene for antibody or the relevant binding domains thereof can be cloned into an expression vector.

As used herein, the term “aptamer” refers to oligonucleic or peptide molecules that can bind to specific antigens of interest and, thus, are also encompassed as macrobiomolecules in the context of the present disclosure. Nucleic acid aptamers usually are short strands of oligonucleotides that exhibit specific binding properties. They are typically produced through several rounds of in vitro selection or systematic evolution by exponential enrichment protocols to select for the best binding properties, including avidity and selectivity. One type of useful nucleic acid aptamers are thioaptamers, in which some or all of the non-bridging oxygen atoms of phophodiester bonds have been replaced with sulfur atoms, which increases binding energies with proteins and slows degradation caused by nuclease enzymes. In some embodiments, nucleic acid aptamers contain modified bases that possess altered side-chains that can facilitate the aptamer/target binding.

Peptide aptamers are protein molecules that often contain a peptide loop attached at both ends to a protein scaffold. The loop typically has between 10 and 20 amino acids long, and the scaffold is typically any protein that is soluble and compact. Peptide aptamers can be generated/selected from various types of libraries, such as phage display, mRNA display, ribosome display, bacterial display and yeast display libraries.

Such affinity reagents, such as the antibodies and antibody derivatives and fragments described above, have great utility to bind antigens of interest to modify activity of the target or provide a detectable label. In combination with the disclosed bifunctional tags, such affinity reagents can be efficiently delivered to bind to antigens in the interior of the cell while preserving cell viability and structure.

In embodiments wherein the macrobiomolecule is a peptide, polypeptide, or protein, the affinity domain comprises a peptide, polypeptide, or protein binding agent. Many binding agents that bind to peptides, polypeptides, and proteins are known and are encompassed by the present disclosure. Persons of ordinary skill in the art can readily select and incorporate such binding agents into the disclosed bifunctional tags. As described above in the context of nucleic acid payloads, the binding agent can be compositions such as stains that are used hereto for to bind and visualize the payload molecule, in this scenario a peptide or protein. An exemplary, nonlimiting example of a molecule that binds protein is Coomassie blue.

The hydrophobic domain is any domain that can interact with a plasma membrane of a cell. In some embodiments, the interaction can lead to insertion, fusion, or otherwise integration into the plasma membrane. In some embodiments, the hydrophobic domain is or comprises a linear or cyclic hydrocarbon structure (e.g., with aromatic rings). In some embodiments, the hydrocarbon structure has between about 6 and 50 carbons. Exemplary categories molecules encompassed by the hydrophobic domain of the present disclosure include sterols (e.g., cholesterol, stigmasterol, ergosterol, and the like), fatty acids (e.g., stearic acid, oleic acid, and the like), glycerolipids (e.g., phosphatidylserine, sphingolipids, and the like), and other hydrophobic chemicals (e.g., parental groups, isobutyl groups, tocopherol, and the like). In some embodiments, the hydrophobic domain is or comprises a steroid, or derivative thereof. In further embodiments, the hydrophobic domain is or comprises cholesterol, or derivative thereof.

The affinity domain and the hydrophobic domain can be conjugated directly to each other without an intervening structure. For example, the direct conjugation can be through a covalent bond. In other embodiments, the affinity domain and the hydrophobic domain are covalently conjugated by a linker domain. Linkers can provide physical separation of the two functional domains (i.e. the affinity domain hydrophobic domain) without substantially interfering with the function of either domain. In some embodiments the linker is flexible to prevent undue steric hindrance of either functional domain. A wide variety of linker domains are known in the art and are encompassed by the present disclosure. A person of ordinary skill in the art can readily an appropriate linker into a disclosed bifunctional tag molecule. In some embodiments, linker domain can provide a stable and relatively permanent linkage. In other embodiments, the linker can be cleavable. Cleavable linkers are known and allow separation of the conjugated domains (here, the affinity domain and the hydrophobic domain) upon the application of certain conditions, including exposure to appropriate enzyme, light, chemical, temperature thresholds, reductive reagents, and the like. In embodiments with cleavable linkers, a tagged macrobiomolecule can pass into the plasma membrane and delivered into the cytosolic side. After the initiation of this process, the linker can be cleaved thereby delivering into the interior of the cell a payload with only the affinity reagent potentially noncovalently attached thereto.

The optional linker can be in a variety of configurations. In some embodiments the linkers linear, connecting a single affinity domain to a single hydrophobic domain. In other embodiments the linker can be branched, permitting multiple iterations of the affinity domain and or the hydrophobic domain within a single tag molecule. For example, a branched linker can provide for conjugation between a single affinity domain and a plurality of hydrophobic domains. Alternatively, a branched linker can provide for conjugation between a plurality of affinity domains and a single hydrophobic domain. Further, a branched linker can provide for conjugation between a plurality of affinity domains and a plurality of hydrophobic domains.

For purposes of illustration, non-limiting examples of linkers include hydrocarbon linkers, aminated linkers, and the like. Exemplary hydrocarbon linkers include pentanes, hexanes, heptanes, octanes, and the like. Exemplary aminated linkers include Tris(2-aminoethyl)amine, 2,2′-Diamino-N-methyldiethylamine, and the like.

As indicated, the affinity domain is configured to noncovalently bind to the macrobiomolecule. The bifunctional tag can be configured to disassociate from the macrobiomolecule upon passage of the macrobiomolecule through the plasma membrane.

In another aspect, the disclosure provides a composition that comprises a combination of one or more bifunctional tags as described above noncovalently bound to a macrobiomolecule, as described above. The macrobiomolecule can be bound by 1, about 5, about 10, about 15, about 20, about 25, about 30, about 35, about 40, about 45, about 50, about 75, about 100 or more bifunctional tags.

In some exemplary embodiments, the bifunctional tag comprises an affinity reagent that is or comprises a nucleic acid binding agent and a hydrophobic domain that is or comprises a steroid or sterol (e.g., cholesterol or derivative thereof). In other exemplary embodiments, the bifunctional tag comprises an affinity reagent that is or comprises a peptide/polypeptide/protein binding agent and a hydrophobic domain that is or comprises a steroid or sterol (e.g., cholesterol or derivative thereof). For example, specific embodiments of the bifunctional tag include: a bifunctional tag that has an affinity domain comprising ethidium (e.g., ethidium bromide) and a hydrophobic domain comprising cholesterol or a derivative thereof; a bifunctional tag that has an affinity domain comprising acridine and a hydrophobic domain comprising cholesterol or a derivative thereof; a bifunctional tag that has an affinity domain comprising doxorubicin and a hydrophobic domain comprising cholesterol or a derivative thereof; a bifunctional tag that has an affinity domain comprising a carbocyanine compound and a hydrophobic domain comprising cholesterol or a derivative thereof; a bifunctional tag that has an affinity domain comprising DAPI and a hydrophobic domain comprising cholesterol or a derivative thereof; a bifunctional tag that has an affinity domain comprising a hoescht dye and a hydrophobic domain comprising cholesterol or a derivative thereof; a bifunctional tag that has an affinity domain comprising distamycin and a hydrophobic domain comprising cholesterol or a derivative thereof; a bifunctional tag that has an affinity domain comprising netropsin and a hydrophobic domain comprising cholesterol or a derivative thereof; a bifunctional tag that has an affinity domain comprising cisplatin and a hydrophobic domain comprising cholesterol or a derivative thereof; a bifunctional tag that has an affinity domain comprising bleomycin and a hydrophobic domain comprising cholesterol or a derivative thereof; and a bifunctional tag that has an affinity domain comprising Coomassie blue and a hydrophobic domain comprising cholesterol or a derivative thereof. Additional embodiments include the bifunctional tags listed immediately above, but with a different sterol, e.g., stigmasterol, ergosterol, and the like, substituted for the cholesterol. Yet more embodiments include the bifunctional tags listed immediately above, but with a fatty acid, e.g., stearic acid, oleic acid, and the like, substituted for the cholesterol. Yet more embodiments include the bifunctional tags listed immediately above, but with a glycerolipid, e.g., phosphatidylserine or sphingolipid, and the like, substituted for the cholesterol. Yet more embodiments include the bifunctional tags listed immediately above, but with a pyrenyl group, substituted for the cholesterol. Yet more embodiments include the bifunctional tags listed immediately above, but with a isobutyl group substituted for the cholesterol. Yet more embodiments include the bifunctional tags listed immediately above, but with a tocopherol substituted for the cholesterol. For example, illustrative substitutions in the recited combination embodiments indicated above can include: a bifunctional tag that has an affinity domain comprising ethidium (e.g., ethidium bromide) and a hydrophobic domain comprising tocopherol; a bifunctional tag that has an affinity domain comprising Coomassie blue and a hydrophobic domain comprising tocopherol; etc.

In another aspect, the disclosure provides a composition comprising a plurality of the bifunctional tags as described herein, a macrobiomolecule, and an acceptable carrier, wherein the plurality of bifunctional tags are non-covalently bound to the macrobiomolecule by their respective affinity domains. The carrier can be acceptable for use in cell culture. Alternatively, the carrier is a pharmaceutically acceptable carrier that can be administered to a subject (e.g., systemic administration).

In another aspect, the disclosure provides a method of delivering a macrobiomolecule to the cytosol of a cell. The method comprises contacting the cell with a macrobiomolecule noncovalently bound by at least one bifunctional tag as described herein or the composition described herein.

Without being bound by a particular theory, the efficacy of the present method using the disclosed bifunctional tags is enhanced by virtue of having one or more bifunctional tags bound to the macrobiomolecule at position(s) along the length, e.g. in the interior, of the macrobiomolecule instead of merely at a terminal end. This allows a plurality of tags to provide a sort of coating to the macrobiomolecule that allows it to interact with the plasma membrane and efficiently enter the cell, whether by direct permeation or by endocytosis. Thus, in some embodiments the macrobiomolecule is bound by a plurality of bifunctional tags. For example, the macrobiomolecule and bifunctional tags are present at a molar ratio of at least about 1:3, at least about 1:4, at least about 1:5, at least about 1:6, at least about 1:7, at least about 1:8, at least about 1:9, at least about 1:10, at least about 1:15, at least about 1:20, at least about 1:25, at least about 1:30, at least about 1:40, or greater.

In some embodiments, the macrobiomolecule has a molecular weight of between about 0.5 kDa and about 150 kDa. For example, in some embodiments macrobiomolecule can be between about 0.5 kDa and about 225 kDa, between about 0.5 kDa and about 200 kDa, between about 0.5 kDa and about 175 kDa, between about 0.5 kDa and about 150 kDa, between about 0.5 kDa and about 125 kDa, between about 0.5 kDa and about 100 kDa, between about 0.5 kDa and about 90 kDa, between about 0.5 kDa and about 80 kDa, between about 0.5 kDa and about 70 kDa, between about 0.5 kDa and about 60 kDa, between about 0.5 kDa and about 50 kDa, 1 kDa and about 225 kDa, between about 1 kDa and about 200 kDa, between about 1 kDa and about 175 kDa, between about 1 kDa and about 150 kDa, between about 1 kDa and about 125 kDa, between about 1 kDa and about 100 kDa, between about 1 kDa and about 90 kDa, between about 1 kDa and about 80 kDa, between about 1 kDa and about 70 kDa, between about 1 kDa and about 60 kDa, between about 1 kDa and about 50 kDa, between about 1 kDa and about 40 kDa, between about 1 kDa and about 30 kDa, between about 1 kDa and about 20 kDa, between about 1 kDa and about 15 kDa, between about 1 kDa and about 10 kDa, 5 kDa and about 80 kDa, between about 5 kDa and about 60 kDa, between about 5 kDa and about 50 kDa, between about 5 kDa and about 40 kDa, between about 5 kDa and about 30 kDa, between about 5 kDa and about 25 kDa, between about 5 kDa and about 20 kDa, and between about 5 kDa and about 15 kDa. As described in more detail below, as the size of the macrobiomolecule increases, e.g. beyond about 50 or 60 kDa, the mode of entry into the cell shifts from direct permeation to endocytosis. Thus, in embodiments where the macrobiomolecule payload is less than about 50 kDa, the macrobiomolecule payload enters the cytosol directly via permeation through the plasma membrane. In many embodiments, at least some, if not the majority, of the bifunctional tags will disassociate as the macrobiomolecule payload enters the cytosol. In this sense, the noncovalently bound bifunctional tags are stripped off of the bifunctional tags during passage through the plasma membrane.

The method can be performed in vitro on cells maintained culture. For example, tagged macrobiomolecules can be added to the culture medium and allowed to enter into the cells. Such applications are useful when delivering, e.g. payloads that can selectively bind to a molecule in the interior of the cell for visualization of a target molecule or to modify the functionality of a target molecule. A benefit is that the cell can remain living. Thus, target molecules can be traced during the dynamic processes occurring in a living cell. In other embodiments, the macromolecule payload can be a nucleic acid leading to modification of the endogenous genome or, in the case of functional RNAi constructs, can modify the functional transcriptional profile of the cell. In some embodiments the cells can be further harvested and formulated for administration to a subject for a cell based therapy.

In other embodiments, the method can be performed on cells in vivo in a subject. Accordingly, these embodiments comprising administering an effective amount of the biomolecules bound by the disclosed bifunctional tags. Typically the biomolecules-bifunctional tag complexes are formulated appropriately for the preferred mode of administration according to ordinary skill in the art. Such applications can encompass any therapeutic method the bifunctional tags facilitate efficient delivery of the desired macrobiomolecules into cytosol the cell. As described below the bifunctional tags can provide for increased efficiency in cytosolic deliver of intact, functional macrobiomolecules. Accordingly, dosing of the therapeutic macrobiomolecule can be reduced compared to the extant delivery methods which are less efficient at achieving final delivery into the cytosol of the cell. As used herein, the term “subject” refers to any individual of any species that is to receive administration of a macrobiomolecule with noncovalently bound bifunctional tags for efficient intracellular delivery. Nonlimiting examples of dental subjects include reptiles and mammals. Exemplary mammals include human, nonhuman primate, mouse, rat, guinea pig, rabbit, cat, dog, sheep, horse, cow, and the like.

### Additional Definitions

Unless specifically defined herein, all terms used herein have the same meaning as they would to one skilled in the art of the present disclosure. Practitioners are particularly directed to Ausubel, F. M., et al. (eds.), Current Protocols in Molecular Biology, John Wiley & Sons, New York (2010), Coligan, J. E., et al. (eds.), Current Protocols in Immunology, John Wiley & Sons, New York (2010), Mirzaei, H. and Carrasco, M. (eds.), Modern Proteomics—Sample Preparation, Analysis and Practical Applications in Advances in Experimental Medicine and Biology, Springer International Publishing, 2016, and Comai, L, et al., (eds.), Proteomic: Methods and Protocols in Methods in Molecular Biology, Springer International Publishing, 2017, for definitions and terms of art.

For convenience, certain terms employed herein, in the specification, examples and appended claims are provided here. The definitions are provided to aid in describing particular embodiments and are not intended to limit the claimed invention, because the scope of the invention is limited only by the claims.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

The terms “subject,” “individual,” and “patient” are used interchangeably herein to refer to a mammal being assessed for treatment and/or being treated. In certain embodiments, the mammal is a human. The terms “subject,” “individual,” and “patient” encompass, without limitation, individuals having cancer. While subjects may be human, the term also encompasses other mammals, particularly those mammals useful as laboratory models for human disease, e.g., mouse, rat, dog, non-human primate, and the like.

The term “treating” and grammatical variants thereof may refer to any indicia of success in the treatment or amelioration or prevention of a disease or condition (e.g., a cancer, infectious disease, or autoimmune disease), including any objective or subjective parameter such as abatement; remission; diminishing of symptoms or making the disease condition more tolerable to the patient; slowing in the rate of degeneration or decline; or making the final point of degeneration less debilitating.

The treatment or amelioration of symptoms can be based on objective or subjective parameters; including the results of an examination by a physician. Accordingly, the term “treating” includes the administration of the compounds or agents of the present disclosure to prevent or delay, to alleviate, or to arrest or inhibit development of the symptoms or conditions associated with disease or condition (e.g., a cancer, infectious disease, or autoimmune disease). The term “therapeutic effect” refers to the reduction, elimination, or prevention of the disease or condition, symptoms of the disease or condition, or side effects of the disease or condition in the subject.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

Illustrative Experimental Disclosure

The following is a description of the design, development, and implementation of a representative bifunctional tag to facilitate cytosolic delivery of RNA macrobiomolecules.

### Introduction

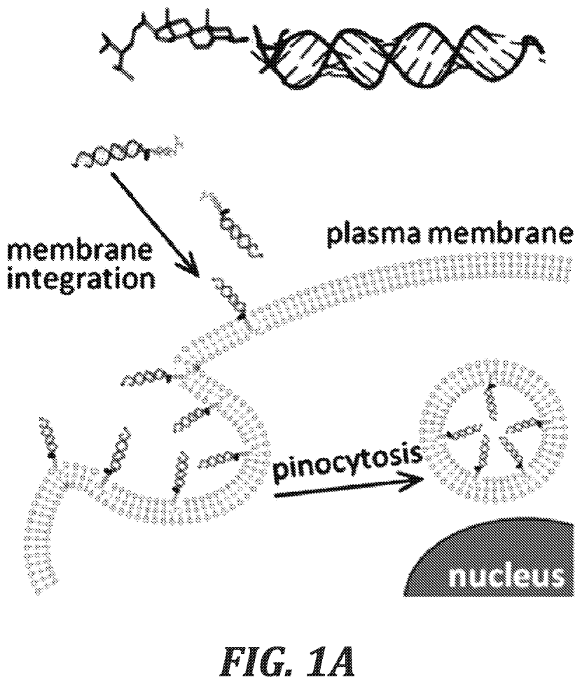

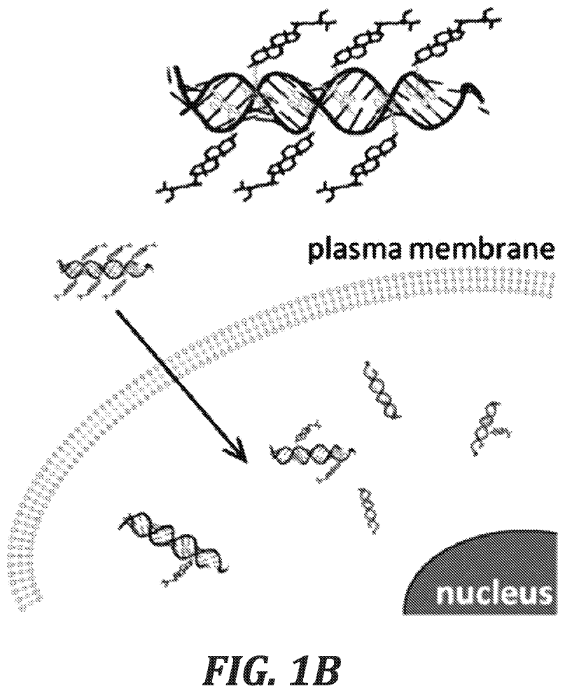

To address the long-standing issue of endosomal sequestration faced in siRNA applications, an alternative concept of tagging siRNAs in a manner to confer membrane-permeant capability was developed. From the observations that (i) siRNAs terminally modified with hydrophobic compounds (di-block amphiphilic structure) have improved association and fusion with cell membranes; and (ii) after the hydrophobic tail fuses into cell membrane, the covalently attached siRNA is immobilized on the cell membrane followed by formation of vesicular structures and cell internalization (but largely stuck in the vesicle, FIG. 1A), it was hypothesized that (i) additional hydrophobic decorations along the double helix (beyond modification of one of the two termini) should further enhance siRNA attraction to cell membrane; and (ii) upon fusion with the cell membrane, siRNA capable of shedding off the hydrophobic compounds might be able to slip into the cytoplasm rather than being stuck on the membrane.

### Results and Discussion

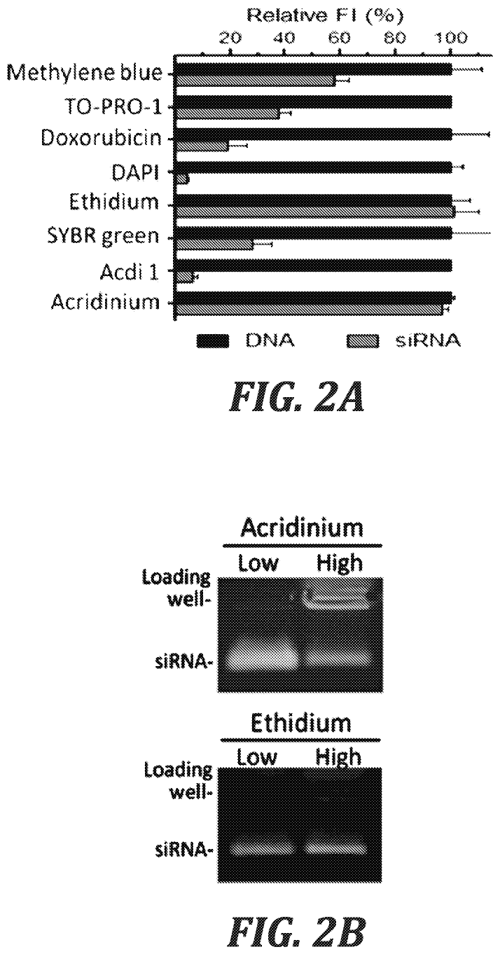

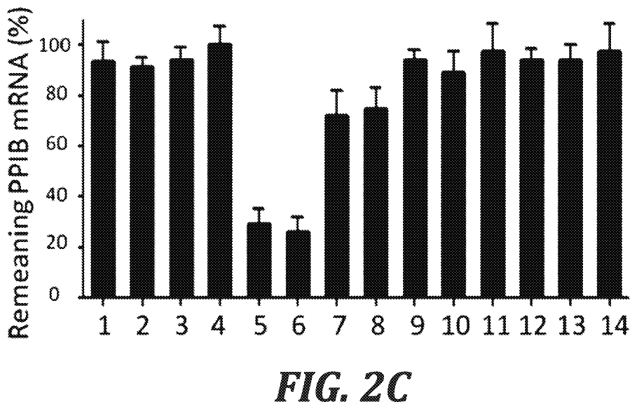

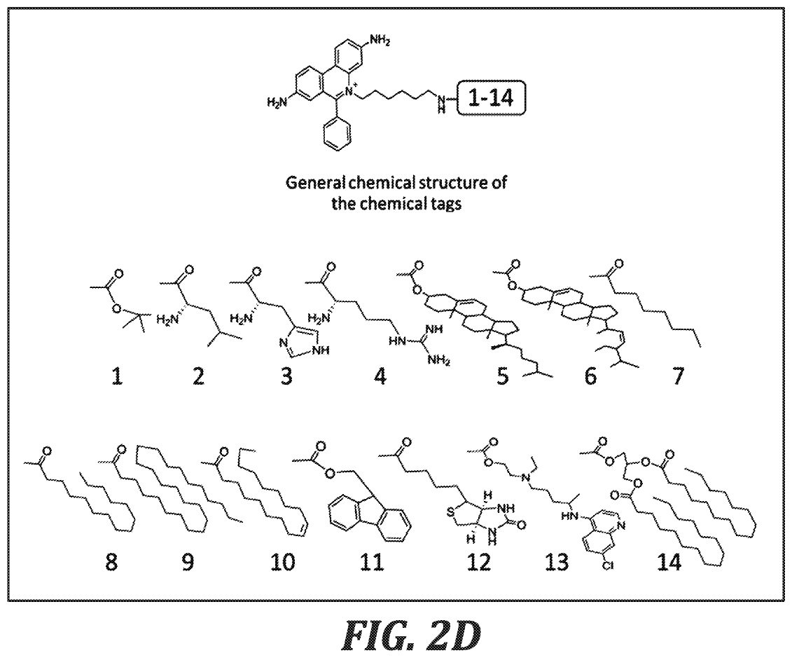

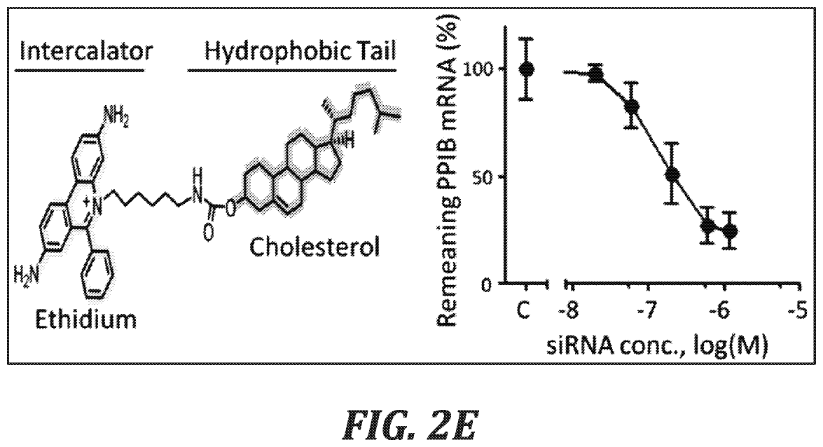

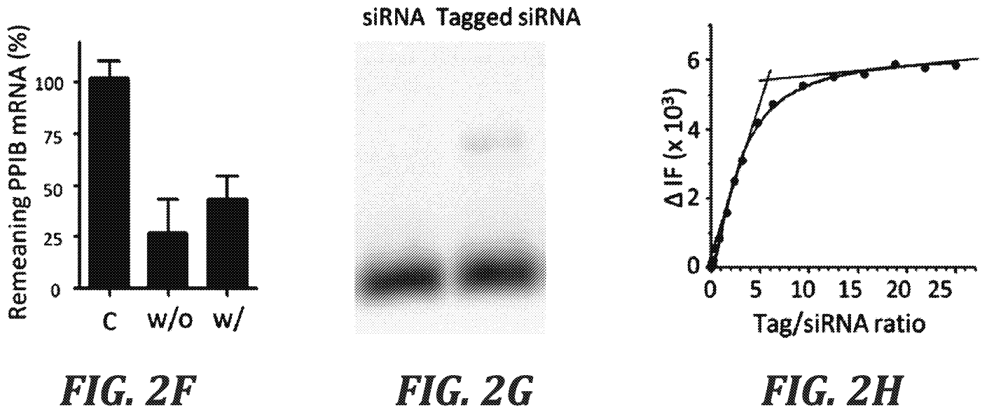

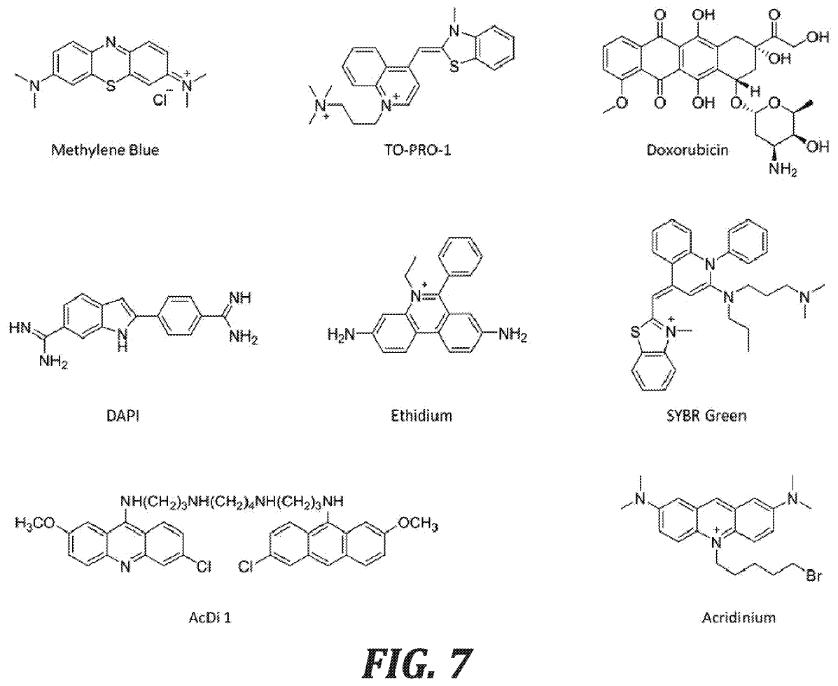

To test this new concept of cytosolic delivery, a conjugation-free methodology was investigated that incorporated small chemical tags to transform siRNA sufficiently hydrophobic, membrane-permeant, and self-deliverable. As shown in FIG. 1B, the small-molecule tags comprise two general functional domains: an affinity group that non-covalently bind with double-stranded RNA (dsRNA) and a hydrophobic moiety capable of fusing with the cell membrane. For a proof of concept, a number of existing oligonucleotide intercalators were tested, due to their potential to non-covalently attach the lipophilic moiety to siRNA. Although DNA intercalating dyes are well-studied and broadly available, selection of dsRNA intercalators is limited. Despite the overall structural similarity, DNA exhibits a standard B-form duplex helix, while dsRNA forms an A-form helix that is ˜20% wider and shorter than the B-form DNA. This conformation difference makes dsRNA exhibit different elastic responses and structural transitions under forces and torques. DNA intercalating dyes apply a stretching force thus may or may not work on siRNA. Eight DNA intercalating dyes (molecular structures shown in FIG. 7) were evaluated utilizing a very convenient feature of these dyes, fluorescence activation upon intercalation into oligonucleotides. By comparing fluorescence intensity increases on both DNA (positive control) and siRNA, two intercalators, ethidium and acridinium, showed similar activation, indicating strong binding with siRNA (FIG. 2A). Further electrophoresis study revealed that acridinium, in addition to intercalation, also unwound the siRNA double helix, forming complexes with the resulting single stranded RNA (emitting red fluorescence at 650 nm, FIG. 2B). Therefore, ethidium was used in the following studies.

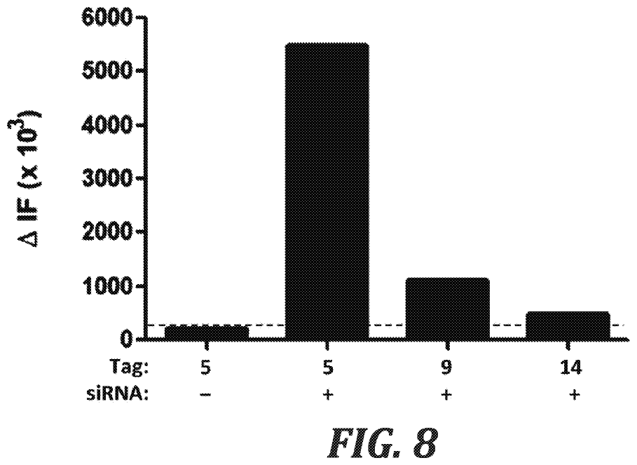

Next, the other building block, the hydrophobic moiety, and its impact on siRNA intracellular delivery was addressed. A small library of 14 chemicals that represent various lipophilicities while sharing a carboxylic acid group for easy conjugation with ethidium was screened. These hydrophobic compounds were chemically linked to ethidium via a hexacarbon (C₆) linker. The resulting small-molecule tags were mixed with a model siRNA sequence against peptidylprolyl isomerase B (PPIB) and tested on a prostate tumor cell line, PC-3, for RNAi. Although the number of tags tested is on the small side, the effect of the hydrophobic moiety on RNAi is quite clear. As revealed in FIGS. 2C and 2D, among the 14 tags, only siRNA complexed with cholesterol-ethidium and stigmasterol (a plant steroid)-ethidium showed significant RNAi effect (quantitatively assessed with real-time PCR). The other tags that feature smaller hydrophobic moieties (tags 1 and 2), cationic or proton-buffering moieties (tags 3, 4, and 13), or short-chain fatty acids (tags 7 and 8) had no or low effect on intracellular siRNA delivery and RNAi. Interestingly, cerotic acid (tag 9) that has a long alkyl chain (C₂₆), similar to cholesterol (C₂₅) and stigmasterol (C₂₅) in the number of saturated carbons, failed to lower the PPIB mRNA level, too. Similarly, a dimer of palmitic acid (1,2-dipalmitate-rac-glycerol, tag 14, C₃₅) also showed no RNAi effect. This result is somewhat surprising since long carbon chain has been previously shown to enhance RNAi efficiency in lipidoid-conjugated siRNA. Further detailed characterization revealed that tag 9 and 14 failed to intercalate into siRNA, likely due to the extremely long and hydrophobic carbon chains that prevent ethidium from binding with siRNA (FIG. 8).

Taken together, these results show that cholesterol, an important constituent of cell membranes, as well as its derivatives can better interact and fuse with cell membrane and carry the siRNA cargo inside cells. A dose-response curve was plotted using the best-performing tag, ethidium-cholesterol. As shown in FIG. 2E, the non-covalently tagged siRNA elicited strong suppression of target gene expression at high nanomolar range (˜74% reduction at 800 nM siRNA). In comparison, it has been well-documented that covalently linked chol-siRNA is only able to knockdown gene expression by ˜30% at similar siRNA concentrations. To further probe whether serum interfere with cell uptake of the tagged siRNA and RNAi, the same experiment was performed in complete cell culture media containing 10% serum. FIG. 2F shows that the RNAi efficiency was only slightly reduced. Gel electrophoresis study revealed that the tagged siRNA has low affinity to albumin and largely remains free in the presence of 2 mg/mL albumin (FIG. 2G).

Another important factor that contributed to the superior RNAi activity of the cholesterol tagged siRNA is the number of cholesterol molecules per siRNA. In contrast to chol-siRNA conjugates where cholesterol can only be linked to one of the two termini of siRNA sequences, the disclosed small-chemical tags can intercalate along the length of the siRNA chain, better masking its high cationic charges and enhancing its interaction with the cell membrane. Previous reports have shown that ethidium can intercalate into DNA at contiguous sites (up to one ethidium every 2 bp). To determine how many ethidium-cholesterol can be tagged onto siRNA, a fluorometric titration experiment probing a series tag/siRNA ratios was carried out. The intersection of the pre- and post-saturation curves provided the stoichiometry of binding ratio of approximately 6:1 (FIG. 2H), which is slightly lower than the theoretical value of 10:1 (one tag every 2 bp). This discrepancy is likely due to steric hindrance caused by the cholesterol molecule. It is important to mention that the at the saturation level of cholesterol tagging, the siRNA remains stable in solution at high concentration. Specifically, a solubility test of the tagged siRNA was performed in PBS at room temperature. Tagged siRNA of various concentrations was suspended in 50 μL of PBS for 24 h followed by high-speed centrifugation. Aggregated siRNA was pelleted to the bottom. Bright field and fluorescence imaging show that the solubility is above 10 μM (not shown). Because of the low molecular weight of siRNA, it is difficult to reliably characterize its size change with or without the tag using dynamic light scattering. Instead, the tagged siRNA was probed with electrophoresis on a 3% agarose gel. The gel was imaged with a Cy5 fluorescence filter set showing the location of siRNA (Cy5-labeled) and alternately with an EtBr fluorescence filter showing ethidium intercalated in siRNA. Agarose gel image shows a nearly negligible migration pattern change, indicating no aggregation or oligomer formation after tagging (not shown).

Upon completion of characterizing the structure and RNAi performance of the tagged siRNA, it was critical to address was how the complex enters the cell cytosol, whether it is a direct traverse of the plasma membrane or through endocytosis. To answer this question, four independent analyses were performed. First, Cy5-labeled siRNA, Cy5-labeled siRNA-cholesterol conjugate and the ethidium-cholesterol tagged siRNA (cy5 labeled) were applied to PC-3 at 37° C. for 6 h and imaged on a confocal microscope. Cell membrane, endosomal vesicles, and cell nuclei were labeled with AlexaFluor 594-conjugated wheat germ agglutinin, Lysotracker, and DAPI, respectively. Overall, the quantity of the noncovalently tagged siRNA inside cells was significantly higher than that of the covalently conjugated (not shown). Additionally, siRNA alone not only failed to enter cells, it did not even associate with the cell membrane, likely due to electrostatic repulsion. In contrast, the tagged siRNA showed bright and even distribution in cytosol. The pattern was apparently unrelated to the endocytosis process, as indicated by the absence of co-localization with the lysosome vesicles. In contrast, the chol-siRNA covalent conjugate exhibited a weak, punctuated intracellular distribution, the characteristic pattern of endocytic pathways.

Second, to rule out the possibility that the tagged siRNA initially enters cells via endocytosis followed by escape from the endosomal vesicles, the cytosolic siRNA fluorescence intensity and distribution at multiple time points were imaged. Briefly, non-covalently tagged siRNA were observed to enter the cytosol in a time-dependent fashion and had a homogeneous distribution at all times; whereas the amphiphilic chol-siRNA first integrated into the cell membrane (0.5 h), followed by a saturable endocytosis process. Fluorescence intensity profiles along a pass in representative cell images were plotted for comparison (not shown). Confocal microscopy showed a time-dependent cell uptake of the tagged siRNA. A striking feature of these micrographs is the homogeneous siRNA cytoplasmic distribution (free of punctate pattern) at all the time points. At early stage of the cell uptake process (e.g., 30 min), although the siRNA fluorescence intensity was low, the distribution was still even, proving the membrane permeant capability of the tagged siRNA. In contrast, the covalently conjugated chol-siRNA was observed to initially bind with cell membrane (30 min), followed by formation of vesicular structures throughout the rest of the imaging period.

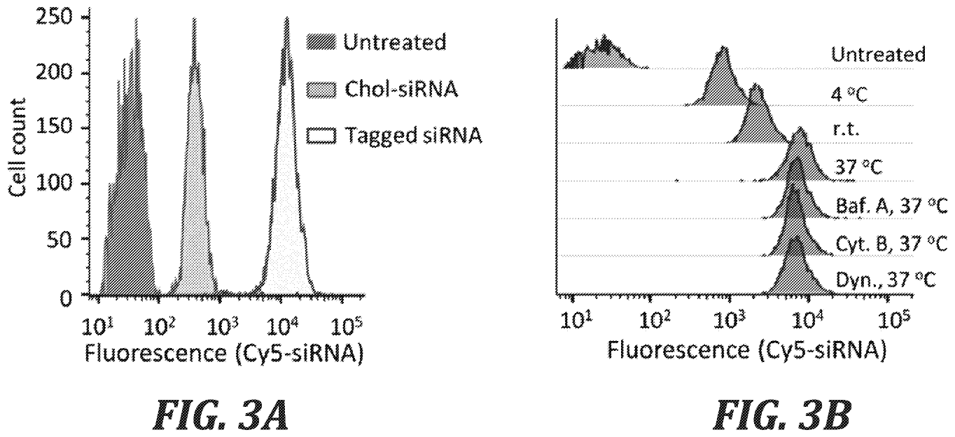

Third, it has been shown that chol-siRNA cell uptake is quick and saturable (within minutes), because it takes several hours for the internalized endocytic vesicle components to be recycled or resynthesized. Qualitative microscopy revealed that cell uptake of the tagged siRNA was completely different. The intracellular fluorescence intensity continuously increased over the entire 6 h of imaging time. In addition, quantitative flow cytometry analysis showed that the tagged siRNA entered cells at significantly higher efficiency than the covalently linked chol-siRNA. Using 2 h incubation as an example, cell uptake of the tagged siRNA is ˜30 folds higher (FIG. 3A).

Lastly, a series of inhibitors that interfere with the endocytosis and pinocytosis processes were explored and studied for their effects on the tagged siRNA's cell entry. Quantitative flow cytometry indicates that cells pre-treated with Bafilomycin A, which blocks vacuolar type H⁺-ATPase, Cytochalasin B that blocks macropinocytosis, or dynasore that affects endocytosis, uptook the tagged siRNA similar to the untreated cells (FIG. 3B). At the same time, dropping the incubation temperature reduced siRNA cell uptake, because cell membrane fluidity and fusion are temperature-dependent (this response is similar to endocytosis). Taken together, these evidences strongly suggest that siRNA non-covalently tagged with ethidium-cholesterol likely enter cells through an initial membrane fusion similar to lipidoid. Because the siRNA is not covalently immobilized on the cell membrane, it can quickly slip into the cytosol, bypassing the endocytosis pathway for improved siRNA cytosolic availability. This hypothesis is strongly supported by the microscopy analyses (not shown) where the tag was used to delivery Cy5-labeled siRNA. Briefly, the fluorescence of ethidium (only fluorescing when bound with RNA) and Cy5 was shown in separate channels, the ethidium channel exhibited stronger fluorescence on the cell membrane than the cytosol, whereas the Cy5 fluorescence was more homogeneous (no clear difference between the membrane and cytosol). This discrepancy indicates that when siRNA enters cells, some tags dissociate and are left behind (trapped in the membrane) due to the non-covalent nature of siRNA-tag binding.

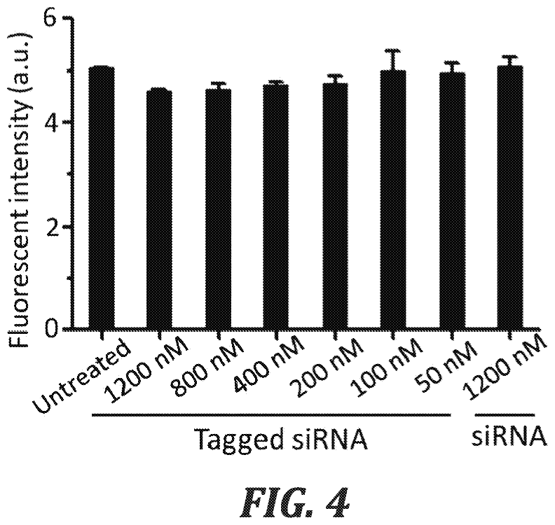

A remaining issue is the potential cytotoxicity of the ethidium-cholesterol tag, in particular whether it damages the membrane integrity, leading to cell death. Because of the low solubility of the tag alone, the tag was first mixed with a siRNA of scrambled sequence and the complex was tested on PC-3 cells at a range of concentrations. No or minimal cytotoxicity was observed even at relatively high concentration (1.2 μM) (FIG. 4). Direct transmembrane delivery avoids saturation of the endocytosis machinery, thus reducing the metabolic burden of the treated cells. It is noteworthy that there have been concerns of ethidium's potential toxicity and mutagenesis effect despite its use as a veterinary medicine at concentrations orders of magnitude higher than solutions used in biology, but it is largely unproven and without direct evidence. Nevertheless, ethidium used here is a model that establishes proof of concept. Any small-molecule alternatives that bind with oligonucleotides should work in a similar fashion.

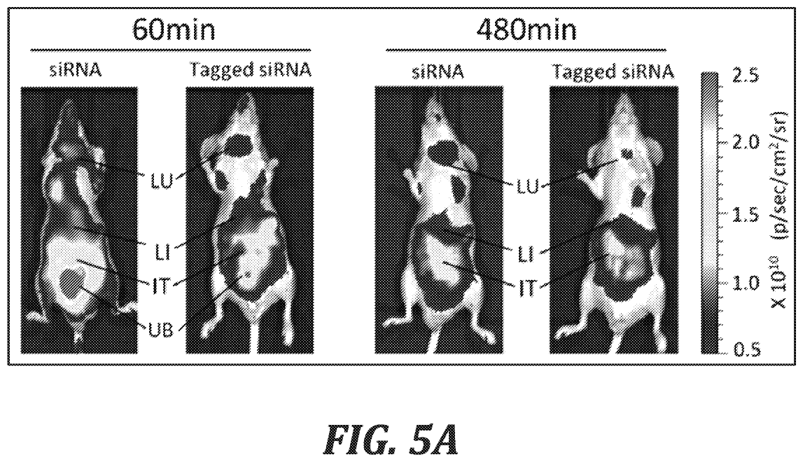

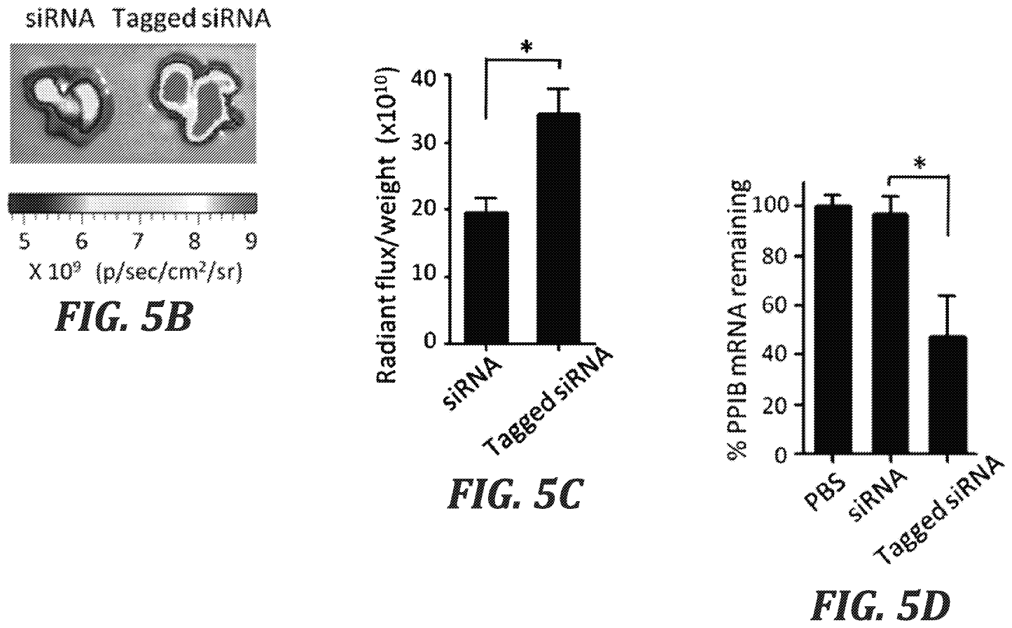

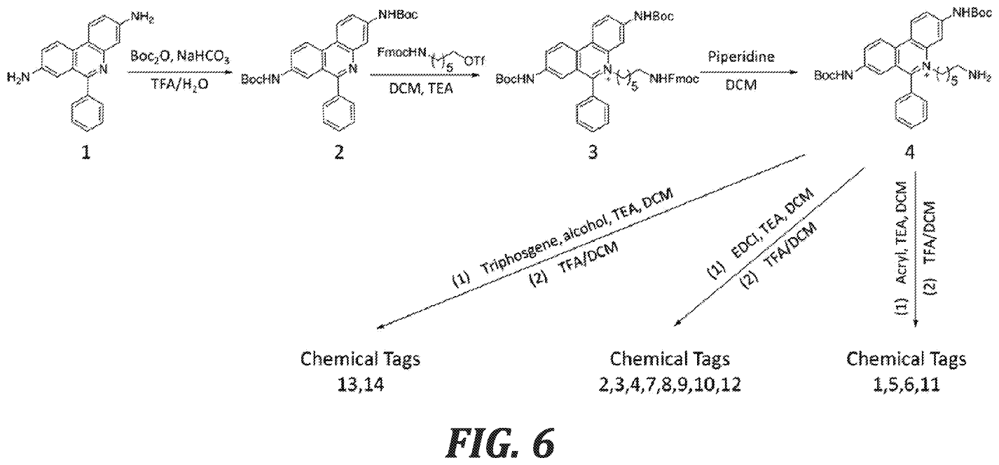

Finally, preliminary animal experiments were conducted to assess whether the tagged siRNA functions in vivo. For biodistribution, siRNA was modified with a NIR dye, Cy5.5. Similar to systemically administered free siRNA, the tagged siRNA was mainly uptaken by the liver with some localization in the kidney and intestines (FIG. 5). More quantitative ex vivo imaging showed that the tagged siRNA had a significant higher accumulation in liver than the free siRNA (FIG. 5B). More importantly, a single intravenous injection of the tagged siRNA led to ˜53% target mRNA (PPIB) reduction whereas the free siRNA showed virtually no effect (FIG. 5C).

### CONCLUSION

RNAi has outstanding potential beyond a research tool in functional genomics and has already started to show major breakthroughs in clinical trials to become a new class of therapeutics. Despite these exciting advances, a longstanding problem remains to be solved. That is, how to delivery siRNA into the cytosol, rather than being trapped and degraded inside endosomes and lysosomes, an effective mechanism resulted from several million years of evolution protecting organisms from invading foreign RNAs. Deviating from the current popular methods based on covalent modification of siRNA, this disclosure describes the development of a new strategy based on bifunctional small molecules that can non-covalently tag siRNA. A number of desirable attributes of this general delivery technology platform that can aid siRNA therapeutic development include (i) simplicity (mix-and-use, free of chemical conjugations) and generalizability (works for all siRNA, sequence independent), (ii) modification of the entire siRNA surface rather than the two termini, potentially allowing fine tuning the level of hydrophobicity, (iii) low cytotoxicity in contrast to transfection reagents, and (iv) most importantly its ability to slip into cytosol avoiding the endocytic sequestration. As a proof-of-principle, we demonstrated transmembrane siRNA delivery by making a series of small chemical tags, in particular ethidium-cholesterol that shows remarkable RNAi in cells. These chemicals were chosen because of their known functionalities in interacting with oligonucleotides and cell membranes, and their commercial availability. Additional embodiments of the individual tag building blocks, the domain that binds to the macrobiomolecule payload, in the domain that interpolates with the plasma membrane, provide even more potent combinations. This development creates a new and exciting avenue for siRNA (and other biologics) research and clinical translation.

### Materials and Methods

Synthesis of the Chemical Tags

The 14 chemical tags were synthesized from 3,8-diamino-6-phenylphenanthridine by N-alkylation with C6 linker and amidization with 14 functional groups. A schematic illustration of the chemical structures for the synthesis of exemplary bi-functional, small molecule tags is provided in FIG. 8. The synthetic route, methods and characterization are described here.

### 3,8-bis(tert-butyloxycarbonylamino)-6-phenylphenanthridine (2)

Compound 2 was synthesized according to a previous report (U.S. Pat. No. 9,035,057, incorporated herein by reference in its entirety). Briefly, one gram (3.5 mmol) of 3,8-diamino-6-phenylphenanthridine (1) and di-tert-butyl dicarbonate (2 g, 9.1 mmol) were dissolved in 200 mL of THF/H₂O (1/1, 100 mL) at room temperature, followed by adding NaHCO₃ powder (1.6 g, 20 mmol). The red mixture was stirred at room temperature for 72 hour. The organic layer was separated and concentrated in vacuo. The crude product was crystalized in petroleum ether/ethyl acetate (1/1) to produce a yellow powder (1.4 g, 85% yield). ¹H NMR (300 MHz, DMSO-d6): δ 1.45 (s, 9H), 1.53 (s, 9H), 7.58 (m, 3H), 7.69 (m, 2H), 7.80 (dd, 1H), 8.0 (d, 1H), 8.20 (dd, 2H), 8.59 (d, 1H), 8.69 (d, 1H), 9.70 (d, 2H). ESI-MS calcd for C₂₉H₃₁N₃O₄ 485.58, found [M+H]⁺ 486.8.

### 3,8-bis(tert-butyloxycarbonylamino)-5-(6-fluorenylmethyloxycarbonylamino-hexyl)-6-phenylphenanthridine (3)

A solution of 6-(fmoc-amino)-1-hexanol (2 g, 6 mmol) in dry DCM (40 mL) was cooled to 0° C., and triflic anhydride (1.4 equiv) and pyridine (1.5 equiv) were sequentially added using syringes. The reaction was stirred for 15 min at 0° C. and then the reaction was monitored by TLC (CHCl₃/MeOH=50/2.5). After completion, the resulting reaction solution was washed with water, 0.3 N HCl, brine and dried with Na₂SO₄. The dried organic solution was concentrated on a rotary evaporator (bath temperature 10° C.). The product, 6-fmoc-hexyl trifluoromethanesulfonate, was collected as a light yellow solid and used directly without further purification.

The triflate (1.5 equiv) described above was added to a solution of 2 (1 g, 2 mmol) in dry DCM at 0° C. The reaction mixture turned red after 2 h and was stirred for 2 days. The reaction solution was washed with water, brine and concentrated in vacuo. The crude product was purified with silica chromatography as a red solid (970 mg, 60% yield). ¹H NMR (300 MHz, DMSO-d6): δ 1.24 (m, 2H), 1.45 (s, 9H), 1.49 (m, 6H), 1.53 (s, 9H), 2.95 (m, 2H), 4.22 (m, 2H), 4.28 (m, 4H), 4.53 (b, 2H), 7.24-7.43 (m, 6H), 7.58-7.89 (m, 8H), 8.17 (d, 1H), 8.37 (d, 1H), 8.63 (s, 1H), 9.00 (dd, 2H), 9.96 (s, 1H), 10.28 (s, 1H). ESI-MS calcd for C₅₀H₅₅N₄O₆ 808.01, found [M+H]⁺ 809.4.

### 3,8-bis(tert-butyloxycarbonylamino)-5-(6-amino-hexyl)-6-phenylphenanthridine (4)

To a room temperature solution of 3 (970 mg) in CHCl₃ (20 mL) was added piperidine (5 mL), and the reaction mixture was stirred at room temperature until the starting material was nearly consumed (˜1 h, monitored with TLC). The reaction was then stopped and the reaction solution was concentrated in vacuo. The red residue was purified with silica chromatography (470 mg red solid, 65% yield). ¹H NMR (300 MHz, DMSO-d6): δ1.30 (m, 2H), 1.42 (s, 9H), 1.49 (m, 6H), 1.55 (s, 9H), 2.77 (m, 2H), 4.35 (t, 2H), 4.55 (b, 2H), 7.67 (m, 3H), 7.79 (m, 3H), 8.18 (d, 1H), 8.38 (dd, 1H), 8.64 (s, 1H), 9.01 (q, 2H), 9.96 (s, 1H), 10.26 (s, 1H). ESI-MS calcd for C₃₅H₄₅N₄O₄ 585.77, found [M+H]⁺ 586.6.

### 3,8-bis(tert-butyloxycarbonylamino)-5-(6-isobutylcarbonylamino-hexyl)-6-phenylphenanthridine (Tag 1)

Compound 4 (50 mg, 0.085 mmol) and isobutyl chloroformate (2 equiv) were sequentially added to a solution of CHCl₃ (5 mL) in the presence of triethylamine (TEA, 2 equiv). The reaction mixture was stirred at room temperature until no starting material was observed (TLC, ˜14 h). The residue obtained upon concentration in vacuo was purified by silica gel chromatography to get the Boc-protected tag 1 (red oily). For Boc de-protection, the purified compound was dissolved in a DCM/TFA mixture (1/1, 5 mL) and stirred at room temperature for 1 h. The solvent was removed and the red residue was purified by silica flash chromatography (CHCl₃/Methol=10/1) as a red solid (33 mg, 80% yield). ¹H NMR (300 MHz, CDCl₃): δ 0.88 (d, 6H), 1.30 (m, 2H), 1.49 (m, 12H), 1.59 (m, 1H), 2.73 (m, 2H), 3.82 (m, 3H), 4.33 (t, 2H), 7.20-7.77 (m, 8H), 8.72 (q, 2H), 9.21 (b, 1H). ESI-MS calcd for C₃₀H₃₇N₄O₂ 485.65, found [M+ 2]²⁺ 244.0.

### 3,8-bis(tert-butyloxycarbonylamino)-5-(6-L-leucinylcarbonylamino-hexyl)-6-phenylphenanthridine (Tag 2)

Boc-L-leucine (43 mg, 0.19 mmol) and N-(3-dimethylaminopropyl)-N′-ethylcarbodiimide (EDC, 1.5 equiv) were sequentially added to a solution of CHCl₃ (5 mL) in the presence of triethylamine (TEA, 2 equiv). The reaction mixture was stirred at room temperature for 5 minutes and compound 4 (40 mg, 0.085 mmol) was added. The reaction was stopped after overnight-stirring. The residue obtained upon concentration in vacuo was purified by silica gel chromatography to produce Boc-protected tag 2 as a red oily compound. For Boc de-protection, the purified compound was dissolved in a DCM/TFA mixture (1/1, 5 mL) and stirred at room temperature for 1 h. The solvent was removed and the red residue was purified by silica flash chromatography (CHCl₃/Methol=10/1) as a red solid (33 mg, 80% yield). ¹H NMR (300 MHz, DMSO-d6): δ0.86 (d, 6H), 1.30 (m, 2H), 1.44-1.67 (m, 13H), 2.72 (m, 4H), 3.87 (m, 2H), 4.33 (t, 2H), 7.20-7.79 (m, 8H), 8.62 (q, 2H), 9.11 (b, 1H). ESI-MS calcd for C₃₁H₄₀N₅O 498.69, found [M+H]⁺ 499.3.

### 3,8-bis(tert-butyloxycarbonylamino)-5-(6-L-histinylcarbonylamino-hexyl)-6-phenylphenanthridine (Tag 3)

Using the similar procedure for tag 2, compound 4 (50 mg, 0.085 mmol) was reacted with the N-Boc-N-trityl-L-histidine (2 equiv) to produce tag 3 as a red solid (18 mg, 40% yield). ¹H NMR (300 MHz, DMSO-d6): δ1.23 (m, 2H), 1.49-1.55 (m, 8H), 1.86 (m, 4H), 2.77 (m, 2H), 3.44 (m, 2H), 4.11 (m, 1H), 4.24 (m, 1H), 4.35-4.43 (m, 4H), 6.28 (d, 1H), 7.20-7.77 (m, 10H), 8.62 (q, 2H). ESI-MS calcd for C₃₁H₃₆N₇O 522.68, found [M+H]⁺ 523.4.

### 3,8-bis(tert-butyloxycarbonylamino)-5-(6-L-argininylcarbonylamino-hexyl)-6-phenylphenanthridine (Tag 4)

Using the similar procedure for tag 2, compound 4 (50 mg, 0.085 mmol) was reacted with the Boc-L-Arg(Pbf)-OH (2 equiv) to produce tag 4 as a red solid (27 mg, 62%). ¹H NMR (300 MHz, DMSO-d6): δ1.31 (m, 2H), 1.49-1.65 (m, 16H), 1.89 (m, 2H), 2.74 (m, 2H), 3.17 (m, 2H), 3.48 (m, 1H), 4.08 (m, 1H), 4.35-4.44 (m, 4H), 7.20-7.77 (m, 8H), 8.96 (q, 2H). ESI-MS calcd for C₃₁H₄₁N₈O 541.72, found [M+H]⁺ 542.6.

### 3,8-bis(tert-butyloxycarbonylamino)-5-(6-cholesterylcarbonylamino-hexyl)-6-phenylphenanthridine (Tag 5)

Using the similar procedure for tag 1, compound 4 (50 mg, 0.085 mmol) was reacted with the cholesteryl chloroformate (2 equiv) to produce tag 5 as a red solid (50 mg, 80%). ¹H NMR (300 MHz, CDCl₃): δ 0.85 (d, 6H), 0.92-1.61 (m, 23H), 1.77-2.33 (m, 4H), 3.15 (m, 2H), 3.28 (t, 2H), 3.90 (b, 1H), 4.48 (b, 1H), 4.62 (m, 2H), 5.36 (m, 1H), 6.30 (s, 1H), 7.25-7.72 (m, 8H), 8.22 (q, 2H). ESI-MS calcd for C₄₄H₅₃ClN₇O₂ 747.40, found [M+H]′ 748.2.

### 3,8-bis(tert-butyloxycarbonylamino)-5-(6-stigmasterylcarbonylamino-hexyl)-6-phenylphenanthridine (Tag 6)

Using the similar procedure for tag 1, compound 4 (50 mg, 0.085 mmol) was reacted with the stigmasteryl chloroformate (2 equiv) to produce tag 6 as a red solid (60 mg, 84%). ¹H NMR (300 MHz, CDCl₃): δ0.70-1.62 (m, 49H), 1.83-2.47 (m, 6H), 3.14 (m, 2H), 3.32 (t, 2H), 4.83 (m, 2H), 5.00-5.55 (m, 3H), 6.44 (s, 1H), 7.25-7.74 (m, 8H), 8.36 (q, 2H). ESI-MS calcd for C₅₅H₇₅N₄O₂ 824.23, found [M+H]⁺ 825.7.

### 3,8-bis(tert-butyloxycarbonylamino)-5-(6-caprylcarbonylamino-hexyl)-6-phenylphenanthridine (Tag 7)

Using the similar procedure for tag 2, compound 4 (50 mg, 0.085 mmol) was reacted with the caprylic acid (2 equiv) to produce tag 7 as a red solid (22 mg, 50%). ¹H NMR (300 MHz, CDCl₃): δ 0.88 (q, 3H), 1.24-1.34 (m, 10H), 1.54-1.62 (m, 10H), 2.52 (m, 2H), 3.41 (m, 2H), 3.80 (m, 2H), 4.39 (b, 2H), 6.32 (s, 1H), 7.31-7.73 (m, 8H), 8.33 (q, 2H), 9.17 (b, 1H). ESI-MS calcd for C₃₃H₄₃N₄O 511.73, found [M+H]⁺ 512.4.

### 3,8-bis(tert-butyloxycarbonylamino)-5-(6-pentadecylcarbonylamino-hexyl)-6-phenylphenanthridine (Tag 8)

Using the similar procedure for tag 2, compound 4 (50 mg, 0.085 mmol) was reacted with the pentadecanoic acid (2 equiv) to produce tag 8 as a red solid (22 mg, 41%). ¹H NMR (300 MHz, CDCl₃): δ 0.87 (q, 3H), 1.24-1.34 (m, 25H), 1.54-1.64 (m, 10H), 2.52 (m, 2H), 3.43 (m, 2H), 3.80 (m, 2H), 4.39 (b, 2H), 6.32 (s, 1H), 7.31-7.73 (m, 8H), 8.33 (q, 2H), 9.14 (b, 1H). ESI-MS calcd for C₄₀H₅₇N₄O 609.92, found [M+H]⁺ 610.6.

### 3,8-bis(tert-butyloxycarbonylamino)-5-(6-cerotylcarbonylamino-hexyl)-6-phenylphenanthridine (Tag 9)

Using the similar procedure for tag 2, compound 4 (50 mg, 0.085 mmol) was reacted with the cerotic acid (2 equiv) to produce tag 9 as a red solid (40 mg, 63%). ¹H NMR (300 MHz, CDCl₃):): δ 0.87 (q, 3H), 1.25-1.36 (m, 47H), 1.51-1.62 (m, 10H), 2.62 (m, 2H), 3.40 (m, 2H), 3.73 (m, 2H), 5.84 (b, 2H), 6.37 (s, 1H), 7.28-7.74 (m, 8H), 8.38 (q, 2H). ESI-MS calcd for C₅₁H₇₉N₄O 764.22, found [M+H]⁺ 765.8.

### 3,8-bis(tert-butyloxycarbonylamino)-5-(6-oleicylcarbonylamino-hexyl)-6-phenylphenanthridine (Tag 10)

Using the similar procedure for tag 2, compound 4 (50 mg, 0.085 mmol) was reacted with the oleic acid (2 equiv) to produce tag 10 as a red solid (30 mg, 53%). ¹H NMR (300 MHz, CDCl₃): δ 0.88 (q, 3H), 1.18-1.31 (m, 20H), 1.54-1.62 (m, 10H), 2.02 (m, 2H), 2.32 (t, 2H), 2.51 (m, 2H), 3.32 (m, 2H), 3.4 (m, 2H), 3.73 (m, 2H), 4.41 (b, 2H), 5.34 (m, 2H), 6.36 (s, 1H), 7.28-7.74 (m, 8H), 8.34 (q, 2H), 9.19 (b, 1H). ESI-MS calcd for C₄₃H₆₁N₄O 649.99, found [M+H]⁺ 651.5.

### 3,8-bis(tert-butyloxycarbonylamino)-5-(6-fluorenylmethyloxycarbonylamino-hexyl)-6-phenylphenanthridine (Tag 11)

Using the similar procedure as tag 1, compound 4 (50 mg, 0.085 mmol) was reacted with the 9-fluorenylmethyl chloroformate (2 equiv) to produce tag 11 as a red solid (49 mg, 95%). ¹H NMR (300 MHz, DMSO-d6): δ1.24 (m, 2H), 1.49 (m, 6H) 2.95 (m, 2H), 4.22 (m, 2H), 4.28 (m, 3H), 4.53 (b, 2H), 7.24-7.43 (m, 6H), 7.58-7.89 (m, 8H), 8.17 (d, 1H), 8.37 (d, 1H), 8.63 (s, 1H), 9.00 (dd, 2H), 9.96 (s, 1H), 10.28 (s, 1H). ESI-MS calcd for C₄₀H₃₉N₄O₂ 607.78, found [M+H]⁺ 608.9.

### 3,8-bis(tert-butyloxycarbonylamino)-5-[6-biotinylcarboylamino-hexyl]-6-phenylphenanthridine (Tag 12)

Using the similar procedure for tag 2, compound 4 (50 mg, 0.085 mmol) was reacted with the biotin-NHS (2 equiv) to produce tag 9 as a red solid (18 mg, 35%). ¹H NMR (300 MHz, DMSO-d6): δ 1.29 (m, 2H), 1.41-1.68 (m, 17H), 2.67-2.79 (m, 7H), 3.12 (m, 1H), 4.16 (m, 1H), 4.29 (m, 1H), 4.35 (m, 2H), 6.33 (s, 1H), 7.31-7.73 (m, 8H), 8.33 (q, 2H), 9.15 (b, 1H). ESI-MS calcd for C₃₅H₄₃N₆O₂S 611.83, found [M+H]⁺ 612.9.

### 3,8-bis(tert-butyloxycarbonylamino)-5-(6-chloroquinecarboylamino-hexyl)-6-phenylphenanthridine (Tag 13)

Carbamate coupling reaction was performed based on a reported procedure with minor modification (A. K. Ghosh, et al., J Am Chem Soc 2006, 128, 5310, incorporated herein by reference in its entirety). Briefly, a solution of 4 (50 mg, 0.085 mmol) and triethylamine (3 equiv) in dry methylene chloride (4 mL) was added slowly over a period of 30 minutes using a syringe pump to a stirred solution of triphosgene (0.35 equic) in methylene chloride (2 mL) at 23° C. After another 5 min of stirring, a solution of hydroxychloroquine (2 equiv) in methylene chloride was added in one portion. The reaction mixture was stirred for 12 hours, diluted with chloroform, washed with water, brine, dried over Na₂SO₄ and concentrated under reduced pressure. The residue was purified by flash chromatography to produce Boc-protected tag 13. For Boc de-protection, the purified compound was dissolved in 5 mL of DCM/TFA (1/1) mixture and stirred at room temperature for 1 h. The solvent was removed and the red residue was purified by silica flash chromatography (CHCl₃/Methanol=10/1) as a red solid (25 mg, 40% yield). ¹H NMR (300 MHz, CDCl₃): δ 1.00 (t, 3H), 1.29 (d, 3H), 1.541-1.72 (m, 12H), 2.49-2.58 (m, 10H), 3.41 (m, 2H), 3.80 (m, 2H), 4.39 (b, 2H), 5.14 (b, 1H), 6.32 (m, 2H), 7.31-7.93 (m, 12H), 8.33 (q, 2H), 8.48 (d, 1H), 9.17 (b, 1H). ESI-MS calcd for C₄₄H₅₃ClN₇O₂ 747.40, found [M+H]⁺ 748.5.

### 3,8-bis(tert-butyloxycarbonylamino)-5-[6-(1,2-dipalmitin)-carboylamino-hexyl]-6-phenylphenanthridine (Tag 14)

Using the similar procedure for tag 13, compound 4 (50 mg, 0.085 mmol) was reacted with the 1,2-Dipalmitoyl-rac-glycerol (2 equiv) to produce tag 14 as a red solid (37 mg, 45%). ¹H NMR (300 MHz, CDCl₃): δ 0.88 (q, 3H), 1.25-1.36 (m, 58H), 1.51-1.62 (m, 12H), 2.35 (m, 2H), 2.77 (m, 2H), 3.40 (m, 2H), 3.73 (m, 2H), 3.75 (m, 1H), 4.30 (m, 1H), 5.09 (m, 1H), 6.35 (s, 1H), 7.28-7.74 (m, 8H), 8.38 (q, 2H). ESI-MS calcd for C₆₁H₉₅N₄O₆ 980.45, found [M+H]⁺ 981.7.

siRNA

2′-O-methylated (underline) and phosphorothioate substituted (*) siRNA sequences were purchased from Integrated DNA Technologies, Inc. (IDT, Inc. Coralville, IW). PPIB sense strand sequence: ACAGCAAAUUCCAUCGUGU*T*T (SEQ ID NO:1); PPIB antisense strand sequence: ACACGAUGGAAUUUGCUGU*T*T (SEQ ID NO:2); thiol-modified sense strand sequence: /5′Thiol-C₆/ACAGCAAAUUCCAUCGUGU*T*T (SEQ ID NO:3). Control siRNA sense strand: GCGCGCUUUGUAGGAUUCG*T*T (SEQ ID NO:4); Control siRNA antisense strand: CGAAUCCUACAAAGCGCGC*T*T (SEQ ID NO:5). The sense strand and antisense strand were mixed at a ratio 1:1 and annealed in duplex annealing buffer (IDT, Inc.) or PBS buffer.

siRNA Labeling

siRNA labeling with cyanine dye at the 5′-end of the sense strand was conducted via thiol-maleimide conjugation. Briefly, the thiol-modified sense strand (40 nmol) was treated with 5 mM tris(2-carboxyethyl)phosphine (TCEP) buffer (pH 8.5) for 10 minutes to cleave the disulfide bond. To the reaction, 3M sodium acetate (10% volume, pH 5.5) was added and the oligonucleotide was precipitated with 3 volume of ethanol. The sense strand pellet was collected by centrifugation and careful removal of the supernatant. The sense oligo was then dissolved in 100 μL PBS, followed by adding 100 μL of Cy5-maleimide (or Cy5.5-maleimide) ethanol solution (10 equiv, ˜400 nmol). After overnight incubation at room temperature, the labeled oligo was precipitated twice with ethanol as described above to remove free cyanine dye. After quantification, the labeled sense strand was annealed with the antisense strand at 1:1 ratio in duplex annealing buffer.

siRNA Delivery and Real Time RT PCR

PC-3 cells were seeded in 24-well plate at density of 5,000 cells per well. After overnight incubation, the media was removed and the cells were treated with 0.5 mL of serum-free media or complete media (10%) containing 800 nM of tagged siRNA (mixing tag 5 with siRNA at ratio of 10/1) for 48 hour. Total RNA was isolated from the cell monolayer using Trizol reagent (Invitrogen) according to the manufacturer's protocol. 200 nanograms of RNA was converted to cDNA using random hexamer primer and MultiScribe Reverse Transcriptase Reagent (Applied Biosystems, Inc., Branchburg, N.J.). 100 nanograms of cDNA was amplified by SensiFast SYBR kit (Bioline, Taunton, Mass.) on a Chromo4™ Real-Time system (Bio-Rad, Hercules, Calif.). The primers used for PPIB amplification were 5′-GATGGCACAGGAGGAAAGAG-3′ (forward primer; SEQ ID NO:6) and 5′-AGCCAGGCTGTCTTGACTGT-3′ (reverse primer; SEQ ID NO:7). Primers for detection of 18s were 5′-GTCTGTGATGCCCTTAGATG-3′ (forward primer; SEQ ID NO:8) and 5′-AGCTTATGACCCGCACTTAC-3′ (reverse primer; SEQ ID NO:9).

Fluorimetric Titration

Fluorescence measurements were made on a Tecan infinite m200 microplate reader. siRNA (0.4 nmol) was mixed with tag 5 of various quantities (from 0 to 10 nmol) in PBS buffer. After 5-minute incubation, the mixture was diluted to 100 μL and the fluorescence was measured with the microplate reader. The fluorescence increment (J. B. Le Pecq, et al., DNA polyintercalating drugs: DNA binding of diacridine derivatives, Proc Natl Acad Sci USA 72(8) (1975) 2915-9; B. Gaugain, et al., DNA Bifunctional intercalators. 2. Fluorescence properties and DNA binding interaction of an ethidium homodimer and an acridine ethidium heterodimer, Biochemistry 17(24) (1978) 5078-88, each of which is incorporated herein by reference in its entirety), ΔIF, is the difference of fluorescence intensity of the tags in the presence or absence of siRNA.

Confocal Imaging

To study the uptake of the non-covalently tagged siRNA and covalently linked chol-siRNA, the intracellular distribution of Cy5-labeled siRNA was monitored on a confocal laser scanning microscope (CLSM, LSM 710, Zeiss). Briefly, PC-3 (5,000 cells) were seeded in a confocal microscope dish (MatTek) one day before the experiments. The cell monolayer was washed with serum-free RPMI 1640 and incubated with the tagged siRNA or chol-siRNA (800 nM) at 37° C. At the indicated time points, endosomes and lysosomes were stained with LysoTracker® Green DND-26 (50 nM) for 30 min (Invitrogen, Grand Island, N.Y.).

Flow Cytometry

The cellular uptake of the tagged siRNA and chol-siRNA is quantified by flow cytometry. Briefly, the tagged siRNA (the siRNA was fluorescently labeled with Cy5, 600 nM) or the chol-siRNA (siRNA was labeled by Cy5, 600 nM) were incubated with cells (˜40,000) for 2 h in RPMI 1640 at 37° C. The cells were washed with PBS and their fluorescence intensity was measured on a LSR II flow cytometer (BD, Franklin Lakes, N.J.). To test the endocytosis inhibition, the cells were pretreated with chemical inhibitors (Bafilomycin A, 150 nM; Cytochalasin B, 50 μg/ml; Dynasore, 40 μM) respectively for 30 minutes at 37° C. The tagged siRNA (Cy5 labeled, 600 nM) was added and incubated with cells in the presence of inhibitors for 2 h at 37° C. The cells were washed and the fluorescent intensity was quantified by flow cytometry.

Solubility Test

The tagged siRNA (mixing tag 5 with siRNA at ratio of 6/1) was suspended in PBS (50 μL) at a series of concentrations (100, 50, 25, 10, 5, 2.5, 1.25 μM). After incubation for 24 h with gentle vortex at room temperature, aggregated siRNA was pelleted by centrifugation at 14,000 g for 10 minutes. Bright-field and fluorescence images were recorded (for fluorescence: ex/em: 530/618 nm, on a Typhoon 7000 scanner, GE healthcare, UK).

Cytotoxicity Study

The cytotoxicity study was performed as previously reported (W. Tai, R. S. et al., Development of a peptide-drug conjugate for prostate cancer therapy, Mol Pharm 8(3) (2011) 901-12, incorporated herein by reference in its entirety). Briefly, PC-3 cells were seeded in 96-well plates at 5,000 cells per well 24 h before the assay. The medium was changed to serum-free medium containing the tagged siRNA (scrambled control siRNA) for a concentration range from 1,200 nM to 50 nM. Untreated cells and cells treated with 1,200 nM of free siRNA were used as the controls. 12 hours later, 10% FBS was added into wells and the culture was incubated for another 36 h. Cytotoxicity was examined using the Alamar Blue-based proliferation assay (Promega Corp. Madison, Wis.) according to manufacturer's protocol.

Gel Electrophoresis and Albumin Binding Assay

Gel electrophoresis was performed on 3% agarose gel at room temperature. Briefly siRNA-Cy5 and the tagged siRNA-Cy5 was loading into the well of 3% agarose gel and electrophoresized at room temperature for 20 minutes under a constant voltage of 120 V. The gel was then imaged using on a Typhoon 7000 scanner (GE healthcare, UK). Two fluorescence filter sets (ex: 637 nm and 530 nm) were used to respectively detect the fluorescence single of label Cy5 (ex: 637 nm) and chelation of siRNA/tag (ex: 530 nm). To evaluate albumin binding, siRNA and tagged siRNA were incubated with BSA (2 mg/mL) at 37° C. for 30 minutes, followed by electrophoresis.

Near-Infrared (NIR) Optical Imaging In Vivo and Liver Gene Knockdown

Male naive athymic nude mice (Strain name, nu/nu) were purchased from Jackson Lab, USA. Animal study protocol was approved by the Institutional Animal Care and Use Committee of University of Washington. Mice were intravenously injected with the tagged siRNA or free siRNA (RNA labeled with Cy5.5) at a dose of 66 nmol/kg. NIR imaging was performed on IVIS Lumina imaging system (Caliper, USA) at 1- and 8-h post injection. After the 8-h imaging experiment, mice were euthanized, and the organs were harvested for ex vivo imaging. Fluorescence intensities were analyzed using Living Image Software and normalized against the tissue mass.

For analysis of gene knockdown, mice were intravenously injected with either PBS, free siRNA or the tagged siRNA at a dose of 120 nmol/kg, and euthanized 72-h post injection. Total mRNA was isolated from the liver and target gene knockdown was quantified using reverse transcription and real time PCR as described above.

The following is a description of a study that expands on the above disclosure to adapt the bifunctional macromolecule tags for use in cytosolic delivery of protein-based macromolecules to the cytosol.

### Introduction

To address the challenge posed by evolved cellular mechanisms to prevent incursion of proteinaceous compounds into cells, a unique, simple solution based on non-covalent cholesterol tagging was designed. This approach was informed by the success in achieving remarkable RNA delivery to the cytosol by membrane permeation (as described above) and enables proteins to directly permeate through the cell membrane without generating transient pores and avoiding endosomal sequestration.

### Results and Discussion

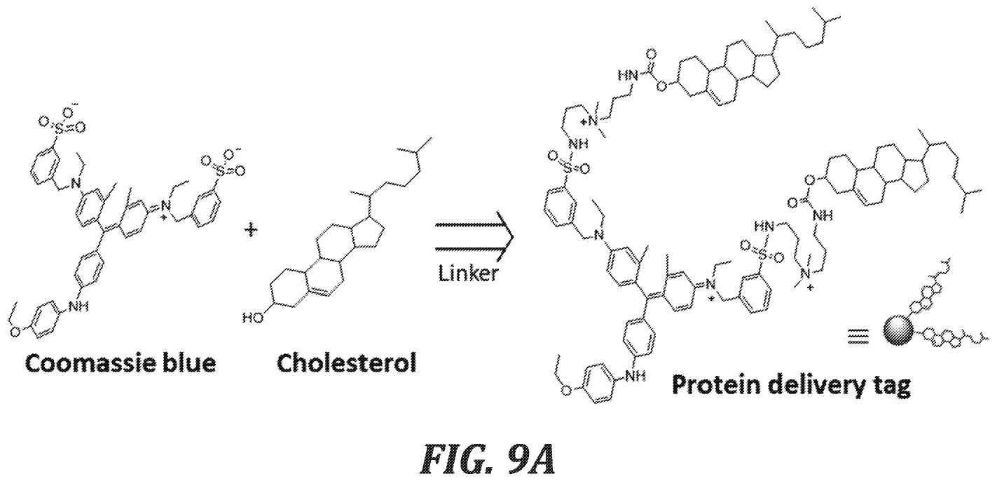

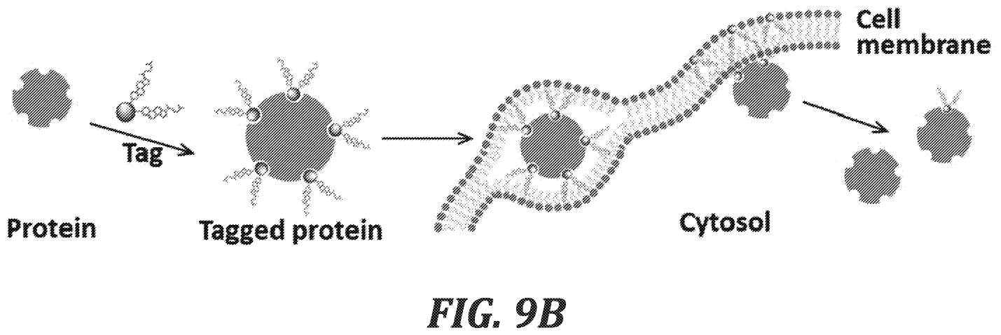

Similar to the above disclosure, a simple small-molecule tag design was implemented by conjugating Coomassie blue (CB) with cholesterol, as shown in FIG. 9A. CB is a protein staining dye commonly used in biochemistry labs. It is also a dye for human use under the trade name Brilliant Peel® (Fluoron GmbH, Ulm, Germany) for retinal surgery, and a drug candidate to treat spinal injuries. CB binds noncovalently with protein surface via the combination of hydrophobic interactions and heteropolar bonding. In parallel, cholesterol is a natural component (˜30% by weight) of all animal cell membranes, essential to maintaining membrane structural integrity and fluidity. To establish proof of concept, two copies of cholesterol and one CB molecule were joined through a short bifunctional linker (the aminated linker increases tag solubility and helps mask the strong negative charges from the sulfonate groups in CB). Upon mixing of the tag with proteins, the CB end of the tag were shown to anchor onto protein molecules independent of the protein sequence, endowing the proteins with a cholesterol-decorated surface compatible with the cell membrane. Similar to transmembrane proteins, the protein-tag complex can insert between the lipid bilayers (FIG. 9B), consistent with the behavior observed on cholesterol covered siRNA macromolecules, as described above. Due to the non-covalent nature of protein-tag interaction, the cholesterol-based tags are eventually pulled away by the cell lipid bilayer. The dissociation leaves behind a hydrophilic protein embedded between the hydrophobic lipid bilayers. Following the like-dissolves-like chemistry rule, the protein molecules are ‘spit’ out of the membrane, slipping directly into the cytosol.

To establish the validity of this technology, proteins of multiple sizes (molecular weight ranging between 6.5 kDa to 150 kDa, namely aprotinin (6.5 kDa), lysozyme (15 kDa), BSA (66 k), and IgG (150 kDa)) were tagged and tested in HeLa cells. It was observed that this unique cytosolic delivery mechanism is selective in protein size, likely determined by the degree of lipid bilayer deformation (bulging). For easy visualization, all proteins were fluorescently labeled prior to exposure to HeLa cells. Confocal fluorescence microscopy revealed distinct intracellular fluorescence patterns of the proteins tested (not shown). Compact proteins were strongly favored for transmembrane delivery. As the protein size increased, the percentage of protein entering cells via direct permeation reduced while via endocytosis increasing. For example, aprotinin with a molecular weight of 6.5 kDa showed bright and homogeneous fluorescence intracellular distribution indicating the absence of endocytosis; lysozyme of 15 kDa generally showed a diffused distribution while having a few punctate bright spots (signature of endosomal sequestration). In comparison, large proteins such as BSA (66 kDa) and IgG (150 kDa) exhibited punctate intracellular fluorescence almost exclusively, showing endocytosis was the dominating cell entry mechanism. As a control experiment, the small protein, aprotinin, without the cholesterol tag was also used to treat cells under the same condition. As expected, only a few punctate fluorescent spots were observed in cells (not shown), showing poor cellular uptake and endosomal escape.

The observed size-effect suggests that the non-covalent cholesterol tagging technology is most suitable for small imaging and therapeutic proteins such as antibody fragments, single-domain antibodies, nanobodies, peptides, and some enzymes. Large proteins that attached to the cell membrane (unlikely to be fully embedded between the lipid bilayers) eventually enter cells through endocytosis. Although the actual protein size cut-off that allows cell membrane encapsulation depends on protein structures because proteins fold into different shapes, thus interacting with the cell membrane differently, the protein size tests suggest that the size cut-off for transmembrane permeation is likely below 60 kDa. Previous studies using rigid inorganic nanoparticles also offer some similar insights. It has been shown that gold nanoparticles coated with dodecanethiol can be embedded between cell lipid bilayers when the particle size is below 5 nm.

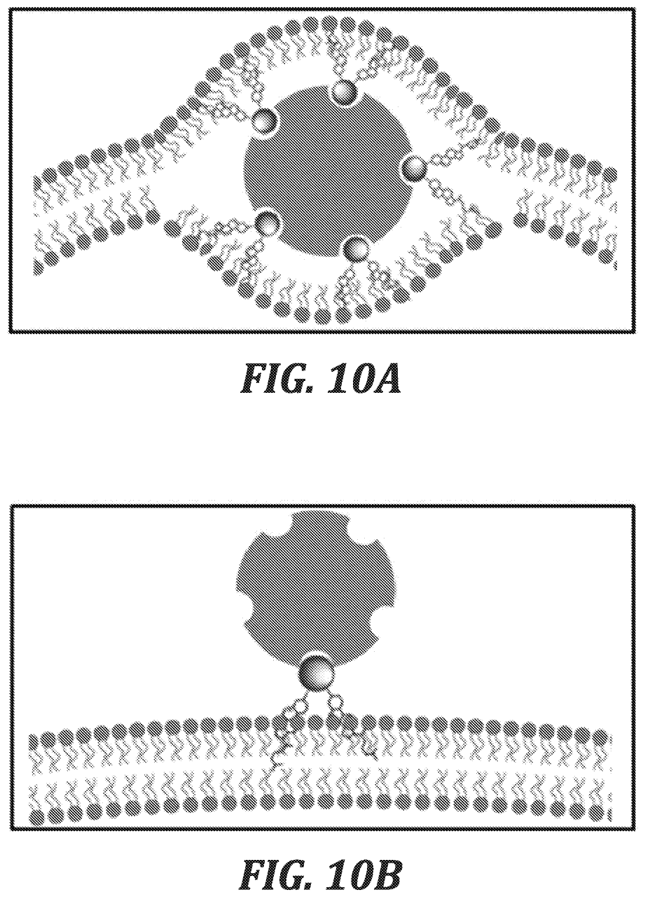

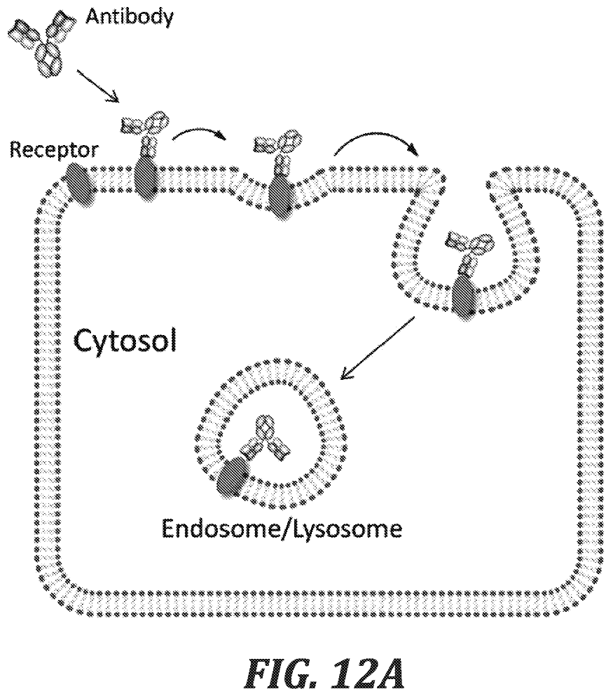

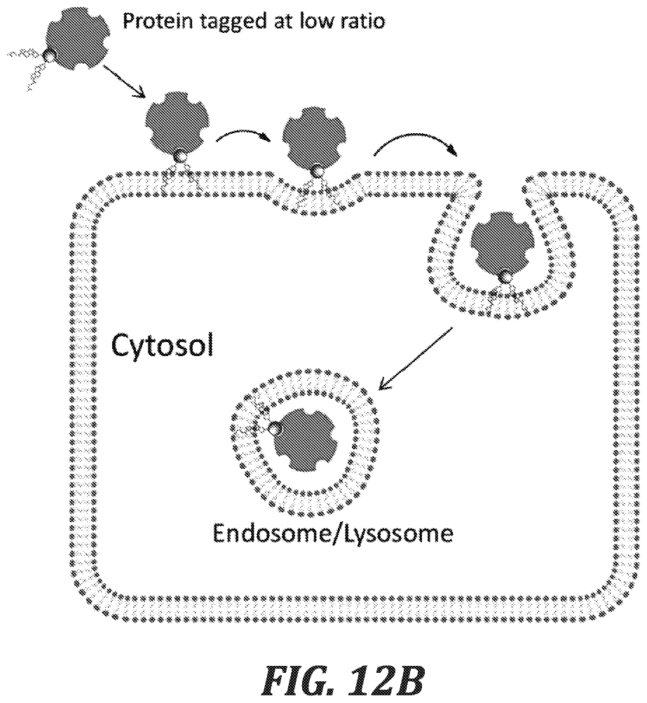

The above confocal microscopy studies demonstrated the effectiveness of the disclosed cytosolic protein delivery technology. To further characterize the cell entry process, it was hypothesized that the cargo proteins should have a certain amount of cholesterol coverage on the surface to be embedded between the bilayer. To probe this effect, the mid-size protein, lysozyme, was mixed with the cholesterol tag at various molecular ratios. Interestingly, at low tag:protein ratios (molar ratio 1:1 and 3:1), only punctate intracellular fluorescence distribution was observed. As the ratio increased to 6:1, diffused fluorescence signal started to appear indicating some contribution from direct membrane permeation, whereas a molar ratio of 8:1 led to significant direct membrane permeation (not shown). This trend revealed the significance of cholesterol density on protein surface for cell membrane permeation. When the density is low, cargo proteins attach to the outer leaflet of the plasma membrane, similar to the way antibodies recognizing cell surface receptors (compare FIGS. 10A and 10B). As a result, the protein molecule enters cells through endocytosis (similar to receptor-mediated endocytosis, FIGS. 12A and 12B).

To further rule out the scenario that the tagged proteins initially enter cells via endocytosis followed by an efficient endosomal escape, the confocal microscopy experiment were performed again using the small protein, aprotinin, at 4° C., a temperature that blocks endocytic membrane trafficking. Without the tag, aprotinin uptake by cells was negligible, whereas, at low tag/protein ratio, aprotinin was virtually confined to the cell membrane (not shown). Remarkably, at the optimal tag/aprotinin ratio, bright and homogeneous intracellular fluorescence was still observed at this low temperature, confirming that the protein-tag complex was not initially taken up by cells via endocytosis. To evaluate the generality of this protein tagging technology, it was tested using nine additional cell types besides HeLa cells (i.e., PC-3, MDA-MB-231, SK-BR-3, DU-145, KB, and HEK-293T). Cells exposed to protein (aprotinin)-tag complex and then stained for protinin and WGA to show the cell boundary. Efficient cytosolic delivery of aprotinin was observed, independent of the cell lines (not shown) indicating that the bifunctional tag promotes cytosolic delivery is not limited to cell-type. Perhaps more importantly, protein transfection directly into the cytosol was also achieved in further experiments of primary cells including human adipose-derived stem cells, primary human dermal fibroblasts, and primary human umbilical vein endothelial cells (HUVEC) (not shown).

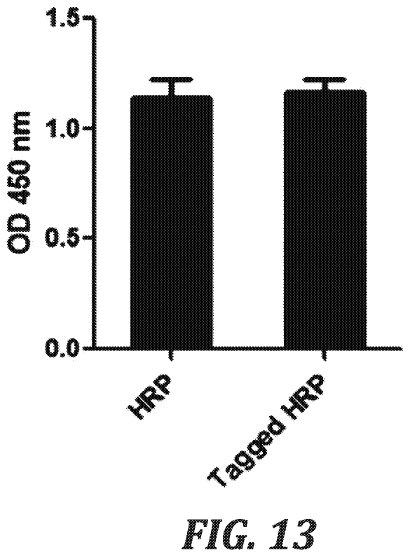

A remaining issue before establishing live-cell intracellular imaging and therapy is whether cholesterol tagging impact protein functions. To probe the effect, two protein molecules, horseradish peroxidase (HRP) and green fluorescence protein (GFP), were tested for functionality after tagging. These two protein models were selected because their functions can be readily measured using optical methods (colorimetric and fluorescent assays, respectively). The fluorescence of GFP before and after cholesterol tagging exhibited no detectable difference (not shown). Similarly, the enzymatic activity of HRP was not affected (FIG. 13). These results demonstrate well-preserved biological functions after cholesterol-tag is adsorbed onto protein surfaces.

Lastly, two applications that should have a major impact on biology and medicine, immunofluorescence in live cells, and delivery of therapeutic proteins inside cells were assessed for compatibility with the disclosed tag approach for cytosolic delivery. Immunofluorescence, one of the most common techniques in biological and medical research, is currently used to profile cell surface markers on live cells (e.g., many flow cytometry studies) missing all the key cell signaling nodes inside cells. Alternatively, immunofluorescence is currently used on fixed cells, missing the temporal dynamics of active cell signaling that determines cell fate, behavior, communications, and growth. To demonstrate immunofluorescence imaging of intracellular biomarkers in live cells, LifeAct, a fusion protein (m.w. ˜30 kDa) of actin-binding peptide and GFP was used because of the distinctive actin structure in cells and because of the significance of actin dynamics. Briefly, HeLa cells were incubated with cholesterol-tagged LifeAct-GFP or LifeAct-GFP alone and the cells were imaged. When LifeAct-GFP was mixed with the cholesterol tag, it not only entered cells but also showed the unique filament structure, indicating preserved LifeAct functionality and labeling specificity. In contrast, LifeAct alone did not show any intracellular labeling (not shown). To confirm the labeling specificity, cells were pretreated with cytochalasin-D before incubating with LifeAct-Tag. The filament structure was completely disrupted (not shown), demonstrate the feasibility of using the disclosed tagging technology in drug response tests.

Additionally, the disclosed tagging technology was compared with Fuse-It-P, a fusogenic liposome, that recommended and provided by the LifeAct-GFP company. Using the same LifeAct-GFP concentration, the disclosed cholesterol tagging technology produced significantly better contrast, whereas the LifeAct-GFP delivered by Fuse-It-P® resulted in some filament structure being observed over a diffused and hazy background. It is also worth mentioning that although, in theory, fusogenic liposomes should directly offload the encapsulated proteins in the cytosol upon fusion with the cell membrane, some liposomes still entered cells via endocytosis, where cargo molecules (LifeAct-GFP in this case) were trapped in endosomes and lysosomes. This sequestration can lead to two unfavorable outcomes, degradation of fluorescent probes which creates diffused hazy background signals, and ultra-bright endosome and lysosome compartments which create dotted/concentrated background inside cells (not shown).

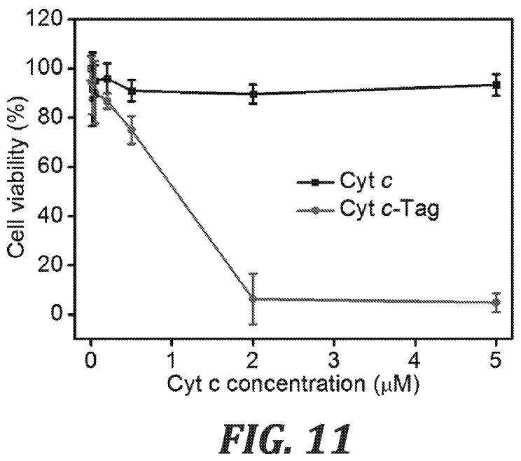

For a proof-of-principle application in therapeutic protein delivery, we selected cytochrome c (Cyt c), an intracellular signaling protein for apoptosis, as a model cargo molecule. To track Cyt c delivery and intracellular distribution, it was labeled with a fluorescent dye (AF488, green). Cell nucleic were counterstained by Hoechst 33258 (blue). Compared to the control (no treatment), cells treated with Cyt c alone showed minor dotted intracellular green fluorescence. This is expected because Cyt c is a highly water-soluble macromolecule (m.w. ˜12 kDa) that cannot diffuse through the cell membrane by itself. Instead, a minute amount of Cyt c was uptaken by cells but trapped in endosomes/lysosomes (not shown). In contrast, the addition of the disclosed cholesterol tag to Cyt c resulted in strong perinuclear fluorescence, likely due to Cyt c binding with intracellular apparatus such as the endoplasmic reticulum (ER), initiating the apoptosis process. Indeed, the cell viability study showed dose-dependent cell death (FIG. 11), similar to previous reports using microinjected Cyt c. As an example, at a Cyt c concentration of 2 μM for cell incubation, no sign of toxicity was detected for cells that were untreated or treated with Cyt c alone while near-complete cell death was found for cells treated with the cholesterol tagged Cyt c (not shown). This study proves the feasibility and potential of specific targeting of certain cell signaling nodes inside cells with protein drugs.

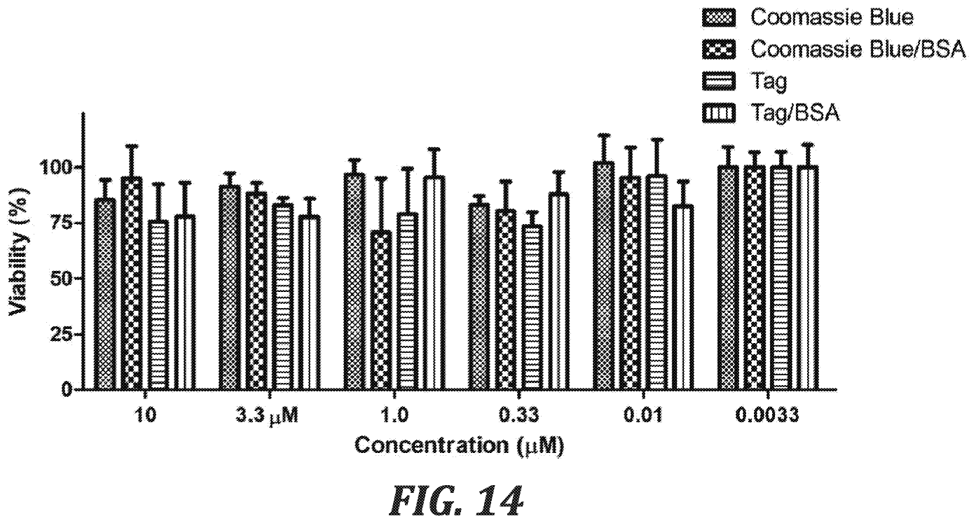

In summary, although DNA and RNA provide the genetic codes, biological structures and functions are mostly realized by proteins and protein interactions. Direct observation, perturbation, modulation, and control of the molecular interactions provide a means for understanding important biological processes and treatment of diseases. For functional protein production, the biology of raising monoclonal antibodies, antibody fragments, nanobodies, and screening for functional peptides has become relatively mature and straightforward that an antibody or peptide can be found for virtually any target molecule. Toward cellular applications, however, the power and potential of functional proteins and peptides are substantially limited. One of the biggest problems is that most signaling nodes and drug targets are inside cells, inaccessible to macromolecular agents. To overcome the barriers of cell membrane and endocytosis that are extremely effective in preventing intact biomolecules to enter the cytosol, an intracellular protein delivery technology was developed based on non-covalent cholesterol tagging. The tag is simple to make by linking Coomassie blue with cholesterol, both of which are biocompatible (FIG. 14); and the technology is broadly applicable to any compact proteins and cell types. In contrast to conventional protein delivery technologies that are mostly based on endocytosis, the disclosed small-molecule tag enables proteins to permeate through the cell membrane without generating pores that cause cytotoxicity. This platform technology opens a whole new realm for cell biology, development of protein therapeutics, and cell engineering.

### Materials and Methods

General

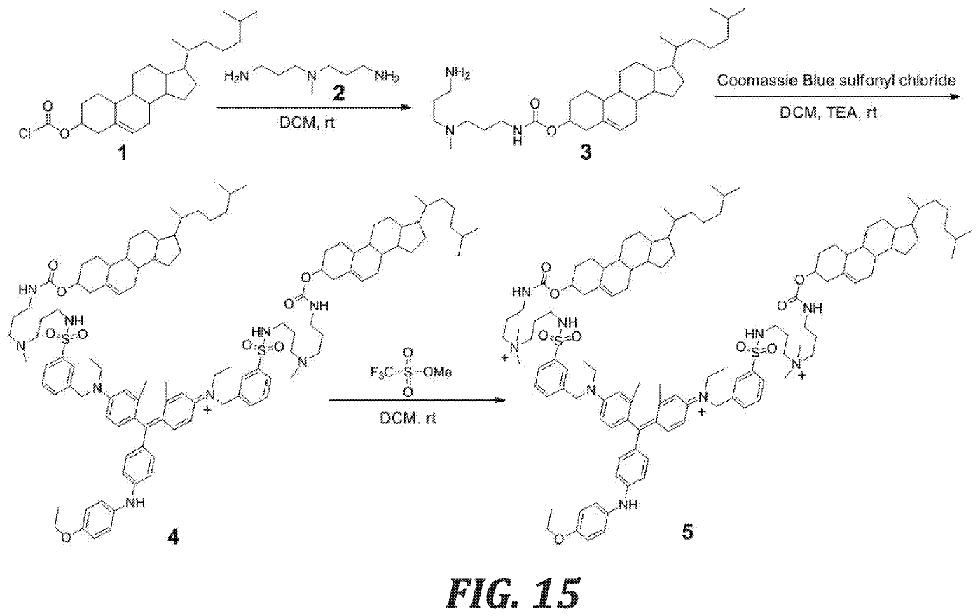

Reference is made to the synthetic route of the CB-cholesterol tag (5) from cholesteryl chloroformate (1), which is illustrated in FIG. 15.

Unless otherwise noted, all organic solvents and chemicals were purchased from Sigma-Aldrich. Cholesteryl chloroformate (1) was purchased from Alfa Aesar. Methyl triflate and phosphorus oxychloride were purchased from TCI America. Coomassie blue G250 was obtained from Chem-impex International. All chemicals were of reagent grade and used as received. LifeAct-GFP and Fuse-It-P® were purchased from ibidi Technology. Cytochrome c (cyt c) was bought from MP Biomedicals. Alexa Fluor® 488 NHS, Cytochalasin D, Hoechst 33258, LIVE/DEAD™ Viability/Cytotoxicity Kit and Microplate BCA Protein Assay Kit were obtained from Fisher Scientific. CellTiter-Blue® Cell Viability Assay was purchased from Promega Corporation. Air and moisture-sensitive manipulation were performed with standard techniques under argon atmosphere. Column chromatography was performed using silica gel 60 (230-400 mesh) from Merck, and thin-layer chromatography was carried out on 0.25 mm Merck silica gel plates (60F-254). NMR spectra were obtained on a Bruker AV301 spectrometer. Chemical shifts are expressed in d (ppm) values, and coupling constants are expressed in hertz (Hz). ¹H spectra were referenced to tetramethylsilane as an internal standard. The following abbreviations are used: s=singlet, d=doublet, t=triplet, quint=quintet, m=multiplet, brs=broad singlet and brd=broad doublet. ESI mass spectra were measured on a Thermo LTQ-OT/Xcalibur 2.0 DS spectrometer.

### Cholestyl 3-(3-aminopropyl methyl amino)propyl carbamate (3)

Compound (2), namely 3,3′-Diamino-N-methyldipropylamine (15 mmol) was dissolved in 30 mL of dry DCM. To the clear solution, 1.35 gram of cholesteryl chloroformate (3 mmol) was slowly added in portions. The reaction was stirred at r.t. for 12 h. After addition of water (30 mL), the organic layer was separated. To completely remove excess compound (2), the organic phase was washed three more times with water. The organic phase was dried with anhydrous Na₂SO₄ and concentrated in vacuo. The product (3) was used in the next step without further purification. (1.3 g, 82% yield). ¹H NMR (300 MHz, CDCl₃): δ 0.68 (s, 3H), δ 0.88 (d, 3H), δ 0.93 (d, 3H), 1.01 (s, 3H), δ 1.04-1.67 (m, 23H), δ 1.75-2.05 (m, 5H), δ 2.17 (s, 3H), δ 2.20 (d, 2H), δ 2.25-2.45 (m, 8H), δ 3.23 (m, 2H), δ 4.49 (m, 1H), δ 5.37 (m, 1H), δ 5.51 (brs, 1H). ESI-MS calcd for C₃₃H₅₉N₃O₂ 529.84, found [M+H]⁺ 530.3.

### Coomassie blue, dicholest carbamyl 3-(3-propyl methyl amino)propyl sulfonamide (4)

Coomassie blue sulfonyl chloride wash synthesized from Coomassie blue G250 according to a previous report (Small 8, 884-891, 2012). Briefly, Coomassie blue G250 (100 mg) was dissolved in 5 mL of dry DMF, followed by 15 mL of dry chloroform. To the solution, 100 μL of phosphorus oxychloride was added drop by drop. The mixture was refluxed for 2 h at 50° C. and then cooled to room temperature (rt). Cold dry ethyl ether (100 mL) was added to the reaction to precipitate the product. The precipitated sulfonyl chloride was collected, washed with ether, dried in vacuo and re-suspended in 10 ml dry DCM. To this suspension, 10 mL of the dry DCM solution of compound (3) (500 mg) was added, followed by 200 μL of triethylamine (TEA). The reaction was stirred at room temperature overnight. The crude product (4) was precipitated out from reaction with ethyl ether, and used in next step without further purification (110 mg, 55% yield).

### Coomassie blue, dicholest carbamyl 3-(3-propyl dimethyl amino)propyl sulfonamide (5)

N-methylation of tertiary amine linker in compound (3) was achieved by powerful methylating agent methyl triflate (MeOTf). Briefly, 50 mg (0.027 mmol) of compound (4) was dissolved in 2 mL of dry DCM, followed by adding MeOTf (50 mg, 0.3 mmol). The reaction was stirred at room temperature for 24 h. After washing with water, the organic layer was separated and concentrated in vacuo. The blue solid was washed with ethyl ether and purified on C-18 chromatography to produce a blue powder (40 mg, 78% yield). ¹H NMR (300 MHz, CDCl₃): δ 0.67 (m, 6H), δ 0.75-1.67 (m, 40H), δ 1.69-2.5 (m, 12H), δ 2.89-3.75 (m, 22H), δ 4.01 (d, 4H), δ 4.41 (m, 6H), δ 5.33 (m, 2H), δ 6.0-7.9 (m, 13H), δ 6.0-7.9 (m, 8H), δ 8.44 (m, 1H), δ11.02 (s, 1H). ESI-MS calcd for C₁₁₅H₁₇₀N₉O₉S₂ 1886.77, found [M+2H]²⁺ 943.9.

Cell Culture

The cancer cell lines were obtained from the American Type Culture Collection (ATCC). The primary cells including human adipose-derived stem cells, primary human dermal fibroblasts, and primary human umbilical vein endothelial cells (HUVEC) were purchased from Zen-bio, Inc. MCF-7 (ATCC# HTB-22), MDA-MB-231 (ATCC# HTB-26), PC-3 (ATCC# CRL-1687), SK-BR-3 (ATCC# HTB-30) and HeLa (ATCC# CCL-2) cells were cultivated in Roswell Park Memorial Institute (RPMI) 1640 medium supplemented with Fetal Bovine Serum (FBS, 10%), penicillin (100 units/mL), and streptomycin (100 μg/mL). DU-145 (ATCC# HTB-81) and HEK-293T (CRL-11268) were cultivated in Dulbecco's Modified Eagle's medium (DMEM) supplemented with FBS (10%), penicillin (100 units/mL), and streptomycin (100 μg/mL). KB cells were cultured in Eagle's minimal essential medium supplemented with FBS (10%), penicillin (100 units/mL), and streptomycin (100 μg/mL). All the primary cells were maintained and plated in dishes with the specific plating media provided by the cell vendor (Zen-bio Inc). All the cell lines were propagated in a humidified 5% CO2 incubator at 37° C.

Synthesis of the Protein Delivery Tag

The cholesterol-based tag was synthesized by conjugating two copies of cholesterol to one CB molecule through an aminated linker, as described herein.

Microscopy

A Zeiss LSM 710 confocal laser-scanning microscope fitted with a plan apochromat objective (40×) was used. Alexa Fluor® 488 was excited with a 488 nm Argon ion laser (5% laser power) and fluorescence was recorded through frame scan. DAPI and Alexa Fluor® 594 were excited with 405 nm and 594 nm laser lines respectively. Bright-field images were recorded with the Differential Interference Contrast (DIC) setting.

Protein Labeling

Proteins were chemically labeled with Alexa Fluor® 488 NHS for imaging and tracking purposes. NETS-activated Alexa Fluor® 488 reacts with the NH₂ group on protein surface, resulting in covalent labeling of dyes on protein surface. Briefly, proteins (pure solid) were dissolved in Dulbecco's phosphate-buffered saline (DPBS or pure water) at 6 mg/mL. Alexa Fluor® 488 NHS dye (Thermofisher) was added in one portion with a reaction ratio of 3/1 (dye/protein molar ratio). The reaction was gently mixed at room temperature overnight followed by purification on PD-10 columns (Bio-Rad). Protein aprotinin was labeled in pure water, instead of DPBS, because of its better solubility in buffers of low ionic strength.

Protein Tagging

Purified Coomassie blue-cholesterol tag powder was reconstituted with ethanol/water (v/v: 4/1) to a concentration of 1.6 mM and stored at 4° C. as a stock. Before tagging, the concentration of all the protein solutions was diluted to 25 μM. To 80 μL of protein solution, 7.5 μL of the tag solution was added. The mixture was quickly mixed by pipetting. The mixture was incubated in a water bath (37° C.) for 8 min and sonicated in an ultrasonic water bath for 30 seconds. After another round of 8-min water bath incubation at 37° C., the protein was ready for delivery.

Cellular Delivery

Cells were seeded on glass-bottom dishes (poly-D-lysine treated, MatTek Corp.) one day before the experiments. The cell monolayer was washed once with serum-free RPMI 1640 media and treated with 2 mL of RPMI 1640 media containing the tagged protein at a final concentration of 1 μM for 2 h. In the end, the cells were counter-stained with Hoechst 33342 and Alexa Fluor® 594 labeled Wheat Germ Agglutinin (WGA-AF594), respectively. The cells were washed three times with RPMI 1640 and imaged on a Zeiss LSM 710 microscope.

To study the delivery effect at low temperature (4° C.), cell monolayers were pre-equilibrated with cold media (RPMI 1640) for 10 minutes at 4° C. and then treated with the cold solution of tagged protein for 3 h. The cells were washed with cold media and imaged immediately.

Toxicity Study

Cytotoxicity of the tag was evaluated on HeLa cells. Briefly, cells were seeded in 96-well plates at 3,000 cells/well. CB, the tag, or tagged protein (BSA) was added at a series of concentrations from 10 μM to 3.3 nM. Untreated cells were used as control. After 48-h incubation, 10 μl of CellTiter-Blue reagent (Promega) was added to the wells and the culture was incubated for another 1 h. Cell viability was examined on a microplate reader (Tecan) at ex/em=560/590 nm and the toxicity was analyzed according to the manufacturer's protocol.

Function Test of the Tagged Protein

Green Fluorescent Protein (GFP, EMD Millipore) was dissolved in water and tagged with the protein delivery tag. The tagged protein was kept at 37° C. for 3 h, then diluted to 100 ng/mL with water. This diluted solution was imaged with a fluorescence imaging system (Light Tools Research, Pasadena, Calif.). The freshly tagged GFP and non-tagged GFP were used as control. The function of tagged Horseradish Peroxidase (HRP, Sigma-Aldrich) was evaluated in a similar way. The enzyme activity was detected by incubating with a TMB substrate solution (Thermofisher) and reading with a Tecan Infinite 200 Microplate Reader.

F-Actin Labeling in Live Cells Using Tagged LifeAct-GFP

HeLa cells were seeded onto glass-bottom dishes. After one-day of culturing, cells were incubated with fresh media containing tagged LifeAct-GFP for 4 h at 37° C., followed by washing and counterstaining Hoechst 33258. The cells were imaged on an Olympus fluorescence microscope equipped with a true-color CCD, Qcolor 5.

Cancer Cell Viability Upon Treatment with Cytochrome c

HeLa cells were seeded onto glass-bottom dishes and cultured overnight. The cells were treated with cyt c (tagged or untagged) of various concentrations in culture media for 4 h, washed, and cultured in fresh media for another 48 h. Cell viability was measured using the dual-color LIVE/DEAD Viability/Cytotoxicity Kit following the manufacturer's instruction.

While illustrative embodiments have been illustrated and described, it will be appreciated that various changes can be made therein without departing from the spirit and scope of the invention.

1. A bifunctional tag, comprising
an affinity domain conjugated to a hydrophobic domain, wherein the affinity domain is configured to non-covalently bind a macrobiomolecule, and wherein the hydrophobic domain is configured to interact with a plasma membrane.

2. The bifunctional tag of claim 1, wherein the macrobiomolecule is or comprises a nucleic acid, and wherein the affinity domain is or comprises a nucleic acid binding agent.

3. The bifunctional tag of claim 2, wherein the nucleic acid is double stranded DNA or double stranded RNA.

4-5. (canceled)

6. The bifunctional tag of claim 3, wherein the double-stranded RNA is a small interfering RNA (siRNA) or a microRNA (miRNA).

7. (canceled)

8. The bifunctional tag of claim 2, wherein the affinity domain is or comprises an intercalating agent.

9. (canceled)

10. The bifunctional tag of claim 1, wherein the macrobiomolecule is or comprises a peptide, polypeptide, or protein, and wherein the affinity domain comprises a peptide, polypeptide, or protein binding agent.

11. The bifunctional tag of claim 10, wherein the polypeptide or protein is an antibody, an antibody fragment or derivative, an enzyme, a cytokine, or a hormone.

12-13. (canceled)

14. The bifunctional tag of claim 11, wherein the antibody is a chimeric antibody, a humanized antibody, or a fragment thereof, or wherein the antibody derivative is a single-chain Fv (scFv) or a single-domain antibody (sdAb or nanobody).

15. (canceled)

16. The bifunctional tag of claim 10, wherein the peptide, polypeptide, or protein binding agent is Coomassie blue.

17. The bifunctional tag of claim 1, wherein the hydrophobic domain is or comprises a linear or cyclic hydrocarbon structure.

18. The bifunctional tag of claim 17, wherein the hydrocarbon has between 6 and about 50 carbons.

19. The bifunctional tag of claim 1, wherein the hydrophobic domain is or comprises a steroid.

20. The bifunctional tag of claim 19, wherein the steroid is cholesterol or a derivative thereof.

21. The bifunctional tag of claim 1, wherein the affinity domain and the hydrophobic domain are covalently conjugated by a linker domain.

22. The bifunctional tag of claim 21, wherein the linker domain is linear and conjugates a single affinity domain to a single hydrophobic domain or the linker domain is branched and conjugates a single affinity domain to a plurality of hydrophobic domains, conjugates a plurality of affinity domains to a single hydrophobic domain, or conjugates a plurality of affinity domains to a plurality of hydrophobic domains.

23-24. (canceled)

25. A composition comprising a plurality of bifunctional tags of claim 1, a macrobiomolecule, and an acceptable carrier, wherein the plurality of bifunctional tags are non-covalently bound to the macrobiomolecule by their respective affinity domains.

26. (canceled)

27. A method of delivering a macrobiomolecule to the cytosol of a cell, comprising contacting the cell with a macrobiomolecule noncovalently bound by at least one bifunctional tag as recited in claim 1, wherein the method is performed in vitro on cultured cells or in vivo in a subject in need thereof.

28. (canceled)

29. The method of claim 27, wherein the macrobiomolecule and bifunctional tags are present at a molar ratio of at least about 1:3.

30. The method of claim 27, wherein the macrobiomolecule has a molecular weight of between about 0.5 kDa and about 250 kDa.

31. (canceled)

32. The method of claim 27, wherein:
the macrobiomolecule is delivered to the cytosol by direct permeation through the plasma membrane, and wherein the macrobiomolecule has a molecular weight of between about 1 kDa and about 50 kDa; or
the macrobiomolecule is delivered to the cytosol by endocytosis, and wherein the macrobiomolecule has a molecular weight of between about 40 kDa and about 250 kDa.

33-39. (canceled)
